(19) United States
(12) Patent Application Publication
(10) Pub. No.: US 2021/0401776 A1
(43) Pub. Date: Dec. 30, 2021

(54) METHOD OF TREATING REFRACTORY EPILEPSY SYNDROMES USING FENFLURAMINE ENANTIOMERS

(71) Applicant: ZOGENIX INTERNATIONAL LIMITED, Berkshire (GB)

(72) Inventor: Parthena MARTIN, Emeryville, CA (US)

(73) Assignee: Zogenix International Limited, Berkshire (GB)

(21) Appl. No.: 17/293,755

(22) PCT Filed: Nov. 20, 2019
(86) PCT No.: PCT/US2019/062432

§ 371 Date: May 13, 2021

Related U.S. Application Data
(60) Provisional application No. 62/854,794, filed on May 30, 2019.
(60) Provisional application No. 62/848,969, filed on May 16, 2019.
(60) Provisional application No. 62/773,846, filed on Nov. 30, 2018.

(51) Int. Cl. A61K 31/137; A61P 25/08; A61K 45/06

(52) U.S. Cl. CPC A61K 31/137; A61K 45/06; A61P 25/08

(57) ABSTRACT

Methods of treating intractable epilepsy syndromes by administering a therapeutically effective dose of a therapeutic agent consisting essentially of a single fenfluramine enantiomer which can be either levofenfluramine or dexfenfluramine, are provided. Intractable epilepsy syndromes for which the present invention finds use include but are not limited to Dravet syndrome, Lennox-Gastaut syndrome, Doose syndrome, West syndrome and refractory seizures. Also provided are methods of treating a neurodegenerative disease in a subject in need thereof. Pharmaceutical compositions for use in practicing the subject methods are also provided.

Methods of treating patients with refractory epilepsy syndromes and symptoms of epileptic encephalopathy syndromes, including Dravet syndrome, Lennox-Gastaut syndrome, Rett syndrome, Doose syndrome and refractory seizures, are described whereby the patient is treated with a therapeutic agent consisting essentially of a single fenfluramine enantiomer, for example dexfenfluramine by itself or as an adjunctive treatment with one or more co-therapeutic agent. Compositions useful in those methods are also disclosed.

### FIELD OF THE INVENTION

The present invention relates to methods of treating refractory epilepsy and symptoms of epileptic encephalopathy syndromes using a therapeutic agent consisting dexfenfluramine or racemic fenfluramine either alone or combination with another sigma-1 receptor agonist, and to pharmaceutical compositions and formulations consisting essentially of the dexfenfluramine enantiomer.

### BACKGROUND OF THE INVENTION

This invention relates to the treatment of refractory epilepsy syndromes, including Dravet syndrome, Lennox-Gastaut syndrome, Doose syndrome and refractory seizure using an amphetamine derivative, specifically fenfluramine.

Fenfluramine, i.e. 3-trifluoromethyl-N-ethylamphetamine, is an amphetamine derivative that is generally a racemic mixture of two enantiomers (RS)—N-ethyl-1-[3-(trifluoromethyl)phenyl]propan-2-amine. Enantiomers, also known as optical isomers, are stereoisomers related to each other by reflection in a plane; i.e., they are non-superimposable mirror images of each other.

| 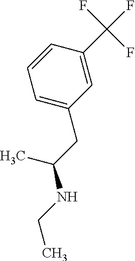 [SMILES: CCN[C@@H](C)Cc1cccc(C(F)(F)F)c1] | 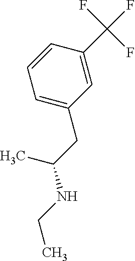 [SMILES: CCN[C@H](C)Cc1cccc(C(F)(F)F)c1] |
|---|---|
| Structure 1 | Structure 2 |
| (2S)-N-ethyl-1-[3-(trifluoromethyl) | (2R)-N-ethyl-1-[3-(trifluoromethyl) |
| phenyl]propan-2-amine | phenyl]propan-2-amine |
| Dexfenfluramine | Levofenfluramine |
| (+)-fenfluramine | (−)-fenfluramine |

Fenfluramine is a chiral molecule that has two enantiomers (Structures 1 and 2 above) dexfenfluramine and levofenfluramine, also referred to respectively as d- and l-fenfluramine, or S- and R-fenfluramine, or (+) and (−)-fenfluramine). Norfenfluramine corresponds to the fenfluramine structure but lacks the ethyl group on the nitrogen atom. The norfenfluramine metabolites retain the stereochemistry of their parent compound and thus (+)-norfenfluramine corresponds with (+)-fenfluramine stereochemistry and (−)-norfenfluramine with (−)-fenfluramine. Racemic fenfluramine was first marketed in the US in 1973 and had been administered in combination with phentermine to prevent and treat obesity. Dexfenfluramine was also marketed in the US for the treatment of obesity. However, in 1997, both fenfluramine and dexfenfluramine were withdrawn from the US market as their use was associated with the onset of cardiac fibrosis and pulmonary hypertension, believed to be caused by the N-ethylated metabolite, norfenfluramine. Subsequently, the drug was withdrawn from sale globally and is at present no longer indicated for use in any therapeutic area; however, low-dose fenfluramine is presently under development for treatment of seizures in Dravet and Lennox Gastaut syndromes.

Despite the health concerns surrounding fenfluramine, attempts have been made to identify further therapeutic uses for that product. Aicardi and Gastaut (New England Journal of Medicine (1985), 313:1419 and Archives of Neurology (1988) 45:923-925) reported four cases of self-induced photosensitive seizures that responded to treatment with fenfluramine.

Clemens, in Epilepsy Research (1988) 2:340-343 reported a study on a boy suffering pattern sensitivity-induced seizures that were resistant to anticonvulsive treatment. Fenfluramine reportedly successfully terminated these self-induced seizures and the author concluded that this was because fenfluramine blocked the photosensitive triggering mechanism.

In Neuropaediatrics, (1996); 27(4):171-173, Boel and Casaer reported on a study on the effects of fenfluramine on children with refractory epilepsy. They concluded that when fenfluramine was administered at a dose of 0.5 to 1 mg/kg/day, this resulted in a reduction in the number of seizures experienced by the patients.

In a letter to Epilepsia, published in that journal (Epilepsia, 43(2):205-206, 2002), Boel and Casaer commented that fenfluramine appeared to be of therapeutic benefit in patients with intractable epilepsy.

Epilepsy is a condition of the brain marked by a susceptibility to recurrent seizures. There are numerous causes of epilepsy including, but not limited to birth trauma, perinatal infection, anoxia, infectious diseases, ingestion of toxins, tumors of the brain, inherited disorders or degenerative disease, head injury or trauma, metabolic disorders, cerebrovascular accident and alcohol withdrawal.

A large number of subtypes of epilepsy have been characterized. For example, the most recent classification system adopted by the International League Against Epilepsy's (“ILAE”) Commission on Classification and Terminology provides the following list of epilepsy syndromes (See Berg et. al., “Revised terminology and concepts for organization of seizures,” Epilepsia, 51(4):676-685 (2010)):

I. Electroclinical Syndromes Arranged by Age at Onset:

A. Neonatal period (1. Benign familial neonatal epilepsy (BFNE), 2. Early myoclonic encephalopathy (EME), 3. Ohtahara syndrome),

B. Infancy (1. Epilepsy of infancy with migrating focal seizures, 2. West syndrome, 3. Myoclonic epilepsy in infancy (MEI), 4. Benign infantile epilepsy, 5. Benign familial infantile epilepsy, 6. Dravet syndrome, 7. Myoclonic encephalopathy in nonprogressive disorders),

C. Childhood (1. Febrile seizures plus (FS+) (can start in infancy), 2. Panayiotopoulos syndrome, 3. Epilepsy with myoclonic atonic (previously astatic) seizures, 4. Benign epilepsy with centrotemporal spikes (BECTS), 5. Autosomal-dominant nocturnal frontal lobe epilepsy (ADNFLE), 6. Late onset childhood occipital epilepsy (Gastaut type), 7. Epilepsy with myoclonic absences, 8. Lennox-Gastaut syndrome, 9. Epileptic encephalopathy with continuous spike-and-wave during sleep (CSWS), 10. Landau-Kleffner syndrome (LKS), 11. Childhood absence epilepsy (CAE));

D. Adolescence —Adult (1. Juvenile absence epilepsy (JAE), 2. Juvenile myoclonic epilepsy (JME), 3 Epilepsy with generalized tonic-clonic seizures alone, 4. Progressive myoclonus epilepsies (PME), 5. Autosomal dominant epilepsy with auditory features (ADEAF), 6. Other familial temporal lobe epilepsies,

E. Less specific age relationship (1. Familial focal epilepsy with variable foci (childhood to adult), 2. Reflex epilepsies);

II. Distinctive constellations: A. Mesial temporal lobe epilepsy with hippocampal sclerosis (MTLE with HS), B. Rasmussen syndrome, C. Gelastic seizures with hypothalamic hamartoma, D. Hemiconvulsion-hemiplegia-epilepsy, E. Other epilepsies, distinguished by 1. presumed cause (presence or absence of a known structural or metabolic condition, then 2. primary mode of seizure onset (generalized vs. focal);

III. Epilepsies attributed to and organized by structural-metabolic causes: A. Malformations of cortical development (hemimegalencephaly, heterotopias, etc.), B. Neurocutaneous syndromes (tuberous sclerosis complex, Sturge-Weber, etc.), C. Tumor, D. Infection, E. Trauma;

IV. Angioma: A. Perinatal insults, B. Stroke, C. Other causes;

V. Epilepsies of unknown cause;

VI Conditions with epileptic seizures that are traditionally not diagnosed as a form of epilepsy per se; A. Benign neonatal seizures (BNS); and B. Febrile seizures (FS).

See Berg et. al, “Revised terminology and concepts for organization of seizures,” Epilepsia, 51(4):676-685 (2010))

As can be seen from, for example, Part V of that list, there are still subtypes of epilepsy that have not yet been fully characterized and thus, the list is far from complete.

Those skilled in the art will recognize that these subtypes of epilepsy are triggered by different stimuli, are controlled by different biological pathways and have different causes, whether genetic or environmental. In other words, the skilled artisan will recognize that teachings relating to one epileptic subtype are not necessarily applicable to other subtypes. This can include recognition that different epilepsy subtypes respond differently to different anticonvulsant drugs.

Dravet syndrome is a rare and catastrophic form of intractable epilepsy that begins in infancy. Initially, the patient experiences prolonged seizures. In their second year, additional types of seizure begin to occur, and this typically coincides with a developmental decline, possibly due to repeated cerebral hypoxia. This leads to poor development of language and motor skills.

Children with Dravet syndrome are likely to experience multiple seizures per day. Epileptic seizures are far more likely to result in death in sufferers of Dravet syndrome; approximately 10 to 15% of patients diagnosed with Dravet syndrome die in childhood, particularly between two and four years of age. Additionally, patients are at risk of numerous associated conditions including orthopedic developmental issues, impaired growth and chronic infections.

Of additional concern, children with Dravet syndrome are particularly susceptible to episodes of status epilepticus (SE). This severe and intractable condition is categorized as a medical emergency requiring immediate medical intervention, typically involving hospitalization. Status epilepticus can be fatal. It can also be associated with cerebral hypoxia, possibly leading to damage to brain tissue. Frequent hospitalizations of children with Dravet syndrome are clearly distressing, not only to the patient but also to family and caregivers.

The cost of care for Dravet syndrome patients is also high, as the affected children require constant supervision and many require institutionalization as they reach teenage years.

At present, although a number of anticonvulsant therapies can be employed to reduce the instance of seizures in patients with Dravet syndrome, the results obtained with such therapies are typically poor and those therapies only effect partial cessation of seizures at best. Seizures associated with Dravet syndrome are typically resistant to conventional treatments. Further, many anticonvulsants such as clobazam and clonazepam have undesirable side effects, which are particularly acute in pediatric patients. Similar challenges exist for treating patients diagnosed with other severe refractory epilepsy syndromes, including but not limited to Lennox-Gastaut, Doose syndrome, West syndrome, Infantile Spasms and refractory seizures.

Polypharmacy, the use of two or more anti-epileptic drugs, for the treatment of refractory epilepsy conditions is currently the treatment regimen most often resorted to. However, it can result in a significant patient burden, as the side effects, or adverse events, from the multiple medications can be additive, and result in limiting the effectiveness of the therapy.

Accordingly, a need remains for providing an improved method for treating or preventing encephalitic encephalopathies, including but not limited to Dravet syndrome, Lennox-Gastaut syndrome, and/or for treating, preventing and/or ameliorating seizures and/or other symptoms experienced by sufferers of epilepsy.

Some potential new treatment options have emerged. Stiripentol is approved in Europe, Canada, Japan and Australia and has only recently been approved in the US, for the treatment of Dravet syndrome. Although it has some anticonvulsant activity on its own, stiripentol acts primarily by inhibiting the metabolism of other anticonvulsants thereby prolonging their activity. It is labeled for use in conjunction with clobazam and valproate. However, the effectiveness of stiripentol is limited, with few if any patients ever becoming seizure free. Further, concerns remain regarding the use of stiripentol due to its inhibitory effect on hepatic cytochrome P450 enzymes.

Fenfluramine has shown considerable promise for treating intractable epilepsy syndromes. Significant reductions in seizure frequency have been observed in both Dravet syndrome and Lennox-Gastaut syndrome patients when low-dose fenfluramine is used as an add-on treatment. See Schoonjans et al., Low-dose fenfluramine significantly reduces seizure frequency in Dravet syndrome: a prospective study of a new cohort of patients, Eur. J. Neurol. 2017 February; 24(2): 309-314 (published online 2016 Oct. 28. (doi:10.1111/ene.13195)); Schoonjans et al., Low-Dose Fenfluramine Significantly Reduces Seizure Frequency in Dravet Syndrome: Update of the Prospective Study (poster presented at the American Epilepsy Society (AES) meeting, Dec. 2-6, 2016, Houston, Tex.); and Ceulemans et al., Successful Use of Fenfluramine as Add-On Treatment for Dravet Syndrome: Update of the Original Patient Cohort (poster presented at the American Epilepsy Society (AES) meeting, Dec. 2-6, 2016, Houston, Tex.). See also Lagae et al., Effectiveness and Tolerability of Low-Dose Fenfluramine (ZX008) in Lennox-Gastaut Syndrome: A Pilot, Open-Label Dose Finding Study (poster presented at the American Epilepsy Society (AES) meeting, Dec. 2-6, 2016, Houston, Tex.).

Investigation of fenfluramine's mechanism of action is ongoing. Previously, preliminary in vitro binding and functional assays revealed that fenfluramine, in addition to acting as a 5HT receptor agonist, also appears to act as a positive allosteric modulator of the sigma-1 receptor. See U.S. patent application Ser. No. 15/717,159, filed on Sep. 25, 2017, the entirety of which is incorporated herein; see also Martin et al., An Examination of the Mechanism of Action of Fenfluramine in Dravet Syndrome: A Look Beyond Serotonin, Abstract ID 239164 (poster presented December 2016 at the American Epilepsy Society Annual (AES) Meeting, Houston, Tex.). In that study, receptors implicated in fenfluramine's mechanism of action as an anti-seizure medication were first identified by means of in vitro receptor binding assays. The activities of racemic fenfluramine, dexfenfluramine and levofenfluramine were then compared in receptor binding, cell- and tissue-function assays. While the investigators observed some differences in the binding and functional activities between dexfenfluramine and levofenfluramine relative to those of racemic fenfluramine, their results, taken together, were consistent with the conclusion that fenfluramine's activity could not be attributed entirely or in significant part to a single enantiomer. However, the activities of the racemic fenfluramine, dexfenfluramine and levofenfluramine were not previously compared directly in an animal models of seizure disorders.

The sigma-1 receptor has been reported to be modulated by fenfluramine as a positive allosteric modulator (See, for example, US 2018/0092864). The sigma-1 receptor is a small (28 kDa), highly conserved, transmembrane protein located in the endoplasmic reticulum (ER) membrane. It is specifically enriched in the ER sub-region contacting mitochondria, called the mitochondrial-associated membrane (MAM). Localization studies also report the sigma-1 receptor at or in i) neuronal nuclear, mitochondrial, and plasma membranes, ii) multiple other CNS cell types (astrocytes, microglia and oligodendrocytes), and iii)CNS-associated immune and endocrine tissues. The varied sites at which sigma-1 receptors are present suggest multiple pathways by which these receptors may influence physiological and pathological processes.

The sigma-1 receptor can migrate between different organellar membranes in response to ligand binding. As chaperone proteins, sigma-1 receptors do not have their own intrinsic signaling machinery. Instead, upon ligand activation, they appear to operate primarily via translocation and protein-protein interactions to modulate the activity of various ion channels and signaling molecules, including inositol phosphates, protein kinases, and calcium channels. The characteristics of sigma-1 interactions in each pathway are still being determined, however, sigma-1 receptor agonists have been identified as providing protection in glutamate mediated interference in learning and memory.

Glutamate is the major excitatory neurotransmitter in the CNS, and its interaction with specific membrane receptors is responsible for many neurologic functions, including learning and memory. Dizocilpine, an antagonist of the N-methyl-D-aspartate receptor has been demonstrated to have in vivo activities which include anesthetic, anticonvulsant, interaction in the brain, neurotoxicity, neuro protection, interaction with abused drugs, motor effects, receptor interaction, behavior, learning and memory. Studies have demonstrated its involvement in working memory processing. Deficits in behavior were noted after administration of the drug and treatment of mice with dizocilpine induced learning impairment. (Maurice T, Hiramatsu M, Itoh J, Kameyama T, Hasegawa T, Nabeshima T. Behavioral evidence for a modulating role of sigma ligands in memory processes. I. Attenuation of dizocilpine (MK-801)-induced amnesia. Brain Res. 1994a; 647: 44-56 and Maurice T, Su T P, Parish D W, Nabeshima T, Privat A. PRE-084, a sigma selective PCP derivative, attenuates MK-801-induced impairment of learning in mice. Pharmacol Biochem Behav. 1994b; 49: 859-69.) Given that refractory epilepsies, including the epileptic encephalopathies, have profound negative effects on cognition and learning, studies of the effect of fenfluramine on sigma-1 function as a dual-mechanism therapeutic are valuable in the development of pharmaceuticals that can treat both seizures and cognitive impairment which occurs either as a sequala to seizures or as a consequence of other pathologies in such epileptic encephalopathic syndromes.

Therefore, the need remains to more fully elucidate the mechanism of fenfluramine's antiseizure effects in mammals for the purpose of achieving a higher standard of care and improving the safety and/or efficacy of fenfluramine when used as an anti-seizure medication or as a dual therapeutic for treatment of seizures and learning and memory impairments in refractory epilepsy and epileptic encephalopathy syndromes.

### SUMMARY OF THE INVENTION

The present invention provides methods of using a therapeutic agent consisting essentially of a single fenfluramine enantiomer for treating seizure related disorders and related symptoms. For example, the disclosed methods are useful in treating patients diagnosed with refractory epilepsy syndromes for which conventional antiepileptic drugs are inadequate, ineffective, or contraindicated, including but not limited to Dravet syndrome, Lennox-Gastaut syndrome, Doose syndrome, Rett syndrome, West syndrome, Infantile Spasms, and refractory seizures. The present invention also provides compositions useful in practicing the methods of the invention, including compositions consisting essentially of an optically pure enantiomer, including optically pure dexfenfluramine, as well as compositions comprising both fenfluramine wherein the first enantiomer is present in a therapeutically effective amount and the second is present in an amount that provides no or insignificant biological effects.

Therefore, in one aspect, the disclosure provides a method of adjunctively treating a patient diagnosed with a disease or disorder by administering a therapeutically effective dose of a therapeutic agent consisting essentially of a single fenfluramine enantiomer or a pharmaceutically acceptable salt thereof to the patient.

In another aspect, the disclosure provides a method of preventing, adjunctively treating, or ameliorating symptoms in a patient diagnosed with a refractory epilepsy syndrome by administering a therapeutically effective dose of a therapeutic agent consisting essentially of a single fenfluramine enantiomer or a pharmaceutically acceptable salt thereof to the patient.

In another aspect, the disclosure provides a method of preventing, adjunctively treating or ameliorating seizures in a patient by administering a therapeutically effective dose of a therapeutic agent consisting essentially of a single fenfluramine enantiomer or a pharmaceutically acceptable salt thereof to the patient, whereby said seizures are prevented, adjunctively treated or ameliorated.

In some embodiments, the single fenfluramine enantiomer is dexfenfluramine. In some embodiments, the single fenfluramine enantiomer is levofenfluramine.

In one aspect, the disease or disorder is a seizure disorder, such as a refractory epilepsy syndrome. In some embodiments, the refractory epilepsy or epileptic encephalopathy syndrome is selected from the group consisting of Dravet syndrome, Lennox-Gastaut syndrome, Doose syndrome, Rett Syndrome, West syndrome, Infantile Spasms, and refractory seizures.

In one aspect, the therapeutic agent is administered in a dosage form selected from the group consisting of oral, injectable, transdermal, inhaled, nasal, rectal, vaginal and parenteral delivery.

In one aspect the therapeutic agent is administered at a daily dose of 120 mg or less, or 60 mg or less, or 30 mg or less. In various embodiments, the dose of the single fenfluramine enantiomer is administered in a dosage form selected from the group consisting of forms for oral, injectable, transdermal, inhaled, nasal, rectal, vaginal and parenteral delivery.

In one aspect, the dose of the single fenfluramine enantiomer is in a range of from 10.0 mg/kg/day to 0.01 mg/kg/day.

In one aspect. the single fenfluramine enantiomer is administered as a monotherapy.

In one aspect, the single fenfluramine enantiomer is co-administered with a second co-therapeutic agent selected from the group consisting of cannabidiol, carbamazepine, ethosuximide, fosphenytoin, lamotrigine, levetiracetam, phenobarbital, progabide, topiramate, stiripentol, valproic acid, valproate, verapamil, and benzodiazepines such as clobazam, clonazepam, diazepam, ethyl loflazepate, lorazepam, midazolam and a pharmaceutically acceptable salt or base thereof. In various embodiments of that aspect, the co-therapeutic agent is one or more agent selected from the group consisting of stiripentol, clobazam, valproate and cannabidiol. In a preferred embodiment, the co-therapeutic agent is stiripentol. In another preferred embodiment, the co-therapeutic agent is cannabidiol. In another preferred embodiment, the single fenfluramine enantiomer is administered in combination with a ketogenic diet regimen. In preferred embodiments, co-administering the single fenfluramine enantiomer with one or more co-therapeutic agents increases blood levels of the single fenfluramine enantiomer by 100% or more relative to fenfluramine blood levels obtained in the absence of the co-administration of the one or more co-therapeutic agent, and/or decreases patient exposure to norfenfluramine.

As shown above and as will be recognized by others skilled in the art, the therapeutic agents provide the important advantage that they are more effective and/or exhibit an improved safety profile as compared to other therapeutic agents and methods currently known in the art.

These and other objects, advantages, and features of the invention will become apparent to those persons skilled in the art upon reading the details of the therapeutic agents and methods of using the same as are more fully described below.

### BRIEF DESCRIPTION OF THE SEVERAL VIEWS OF THE DRAWING(S)

The invention is best understood from the following detailed description when read in conjunction with the accompanying drawings. Included in the drawings are the following figures:

FIG. 1 shows two graphs of data from a zebrafish locomotor assay comparing varying concentrations of fenfluramine enantiomers in scn1Lab−/− mutants and wild type zebrafish: 1A shows results with (+)-fenfluramine and 1B shows results with (−)-fenfluramine.

FIG. 2 shows two graphs of data from a zebrafish locomotor assay comparing varying concentrations of norfenfluramine enantiomers in scn1Lab−/− mutants and wild type zebrafish: 2A shows results with (+)-norfenfluramine and 2B shows results with (−)-norfenfluramine.

In FIGS. 1 and 2, statistically significant differences are indicated as *p<0.05, **p<0.01 and ****p<0.001.

FIG. 3 shows four graphs of data from another zebrafish locomotor assay comparing a fixed concentration of fenfluramine and norfenfluramine enantiomers in scn1Lab−/− mutants and wild type zebrafish: 3A shows results with (+)-fenfluramine, 3B shows results with (−)-fenfluramine, 3C shows results with (+)-norfenfluramine and 3D shows results with (−)-norfenfluramine. 3E is a summary in cartoon form of the assay performed using an optical measuring device measuring movements of zebrafish larvae in 96-well plates. Further detail is provided in Example 1. Scn1Lab−/− mutants in vehicle exhibit much increased movement in comparison to the wild type.

FIG. 4 shows four graphs of data from a zebrafish assay measuring the frequency of epileptiform events using fixed concentrations of fenfluramine and norfenfluramine enantiomers in scn1Lab^{-/-} mutants and wild type zebrafish: 4A shows results with (+)-fenfluramine, 4B shows results with (−)-fenfluramine, 4C shows results with (+)-norfenfluramine and 4D shows results with (−)-norfenfluramine. 4E shows images from the assay performed using an microscope and electrode positioning device for inserting an electrical sensing probe into the brain of a zebrafish larvae. Further detail is provided in Example 1.

FIG. 5 shows four graphs of data from a zebrafish assay measuring the cumulative duration of epileptiform events using fixed concentrations of fenfluramine and norfenfluramine enantiomers in scn1Lab^{-/-} mutants and wild type zebrafish: 5A shows results with (+)-fenfluramine, 5B shows results with (−)-fenfluramine, 5C shows results with (+)-norfenfluramine and 5D shows results with (−)-norfenfluramine. Further detail is provided in Example 1.

In FIGS. 3,4 and 5, statistically significant differences are indicated as * p<0.05, ** p<0.01, *** p<0.001 and **** p<0.0001.

FIG. 6: 6A summarizes data in six graphs on racemic fenfluramine and fenfluramine enantiomers on dizocilpine-induced learning deficits as measured in the Y-maze and passive avoidance assays. 6B presents data in six graphs on racemic norfenfluramine and norfenfluramine enantiomers on dizocilpine-induced learning deficits as measured in the Y-maze and passive avoidance assays. Graphs show mean±SEM (vertical line) in FIGS. 6A and 6B (a, c, e) and median (black bar) and interquartile range (shaded bar) in FIGS. 6A and 6B (b, d, f).

FIG. 7: 7A summarizes data in four graphs on the combination of PRE-084 and racemic fenfluramine on dizocilpine-induced learning deficits as measured in the Y-maze and passive avoidance assays. Within 7A, labels (b) and (d) indicate synergistic combinations with “S.” 7B summarizes data in four graphs on the combination of PRE-084 and (+)-fenfluramine on dizocilpine-induced learning deficits as measured in the Y-maze and passive avoidance assays. Within 7B, labels (b) and (d) indicate synergistic combinations with “S.”

FIG. 8 shows four graphs that both norfenfluramine enantiomers antagonize the effect of PRE-084 in dizocilpine-treated mice: (a, b) spontaneous alternation and (c, d) passive avoidance. Graphs show mean±SEM (vertical line) in (a) and median (black bar) and interquartile range (shaded bar) in (c).

FIG. 9 includes two graphs that summarize the effects of racemic fenfluramine (abbreviated herein as “FA,” “FFA” or “FEN”) treatment in 6-Hz mice, as described in Example 2. FIG. 9A is a bar graph showing the percentage of animals protected for mice treated with vehicle, with 20 mg FA, and with 5 mg/kg FA. FIG. 9B shows the effects on seizure duration. **p<0.01 and ***p<0.001 vs. vehicle (VHC)-injected; n=6-10 NMRI mice for all experimental conditions. Protection being defined as the absence of a seizure within the expected time frame (i.e. below the minimum seizure duration in VHC treated mice).

FIG. 10(a-f): shows studies of the combination of PRE-084 and (−)-fenfluramine on dizocilpine-induced learning deficits, as measured by spontaneous alternation performance in the Y-maze (a, b) and step-through latency in the passive avoidance test (c, d).

FIG. 11: presents combination studies between DHEAS and Fenfluramine in dizocilpine-treated mice: spontaneous alternation performance in the Y-maze (a, b) and step-through latency in the passive avoidance test (c, d).

FIG. 12: presents combination studies between PREGS and Fenfluramine or (+)Fenfluramine in dizocilpine-treated mice: spontaneous alternation performance in the Y-maze (a, b) and step-through latency in the passive avoidance test (c, d).

### DETAILED DESCRIPTION OF THE INVENTION

Before the present compositions and methods are described, it is to be understood that this invention is not limited to the particular formulations and methods described, as such can, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges can independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those include limits are also included in the invention.

The publications discussed herein are incorporated herein by reference to disclose and describe the methods and/or materials in connection with which the publications are cited. They are provided solely for their disclosure prior to the filing of the present application. Nothing herein is to be construed as an admission that the present invention is not entitled to antedate such publication by virtue of prior invention. Further, the dates of publication provided may be different from the actual publication dates which may need to be independently confirmed.

Although any methods and materials similar or equivalent to those described herein can be used in the practice or testing of the present invention, the preferred methods and materials are now described.

### Definitions

It must be noted that as used herein and in the appended claims the singular forms “a,” “an,” and “the” include plural referents unless the context clearly dictates otherwise. Thus, for example, reference to “a formulation” includes a plurality of such formulations and reference to “the method” includes reference to one or more methods and equivalents thereof known to those skilled in the art, and so forth.

Unless defined otherwise, all technical and scientific terms used herein have the same meaning as commonly understood by one of ordinary skill in the art to which this invention belongs.

By “therapeutically effective amount” is meant the concentration of a compound that is sufficient to elicit the desired biological effect (e.g., treatment or prevention of epilepsy and associated symptoms and co-morbidities, including but not limited to seizure-induced sudden respiratory arrest (S-IRA)).

To avoid doubt, the term “prevention” of seizures means the total or partial prevention (inhibition) of seizures. Ideally, the methods of the present invention result in a total prevention of seizures. However, the invention also encompasses methods in which the instances of seizures are decreased in frequency by at least 30%, at least 40%, at least 50%, at least 55%, at least 60%, at least 65%, at least 70%, at least 75%, at least 80%, at least 85%, or at least 90%. In addition, the invention also encompasses methods in which the instances of seizures are decreased in duration or severity by at least 30%, at least 40%, at least 50%, at least 55%, at least 60%, at least 65%, at least 70%, at least 75%, at least 80%, at least 85%, or at least 90%.

The terms “fenfluramine enantiomer” and “enantiomer” are used to indicate one of levofenfluramine and dexfenfluramine without distinguishing between them.

The term “pure fenfluramine enantiomer” or “pure enantiomer composition” is used to indicate optically pure levofenfluramine or dexfenfluramine. A “pure enantiomer composition” is one wherein only an optically pure fenfluramine enantiomer, such as optically pure levofenfluramine or optically pure dexfenfluramine, is present.

The terms “single fenfluramine enantiomer” and “single enantiomer” refer to an enantiomer that can be but is not necessarily optically pure. A “single enantiomer composition” as used herein to encompass pure enantiomer compositions and also compositions that consist essentially of a first fenfluramine enantiomer, for example levofenfluramine or dexfenfluramine, with the second enantiomer being present in an amount that is insufficient to have any or significant biological or therapeutic effects, such that any observable biological activity or therapeutic effect is attributable either entirely or substantially to the first fenfluramine enantiomer. Enantiomerically enriched mixtures are often expressed as a percentage of the predominant stereoisomer and referred to as “enantiomeric excess” or “enantiomeric enrichment”.

### Overview of the Invention

Prior to the inventor's work, there was little information about fenfluramine's mechanism of action or its biological activity outside of its use as a weight loss drug. It was known to be a 5-HT receptor agonist, and to increase serotonin transmission, by inhibiting the serotonin reuptake pump and stimulating release of serotonin from the synaptosomes. However, the differences between the two enantiomers' pharmacokinetics and activity were not extensively studied except in the context of fenfluramine's use as an anorectic. The mean elimination half-lives of fenfluramine's enantiomers was known to differ (19 hours for dexfenfluramine and 25 hours for levofenfluramine, respectively). More recently, research shedding light on the mechanism of action demonstrated that there was little difference between racemic fenfluramine and the two enantiomers in studies of receptor binding and cell- and tissue-function. (See P Martin et al., “An Examination of the Mechanism of Action of Fenfluramine in Dravet Syndrome: A Look Beyond Serotonin” poster presentation at the 7^{th} Annual American Epilepsy Society, Dallas, Tex., and Abstract ID 239164 in the corresponding program book, e-published Nov. 21, 2016 online at [issuu.com/americanepilepsysociety/docs/aes_program_11-22-16_web]).

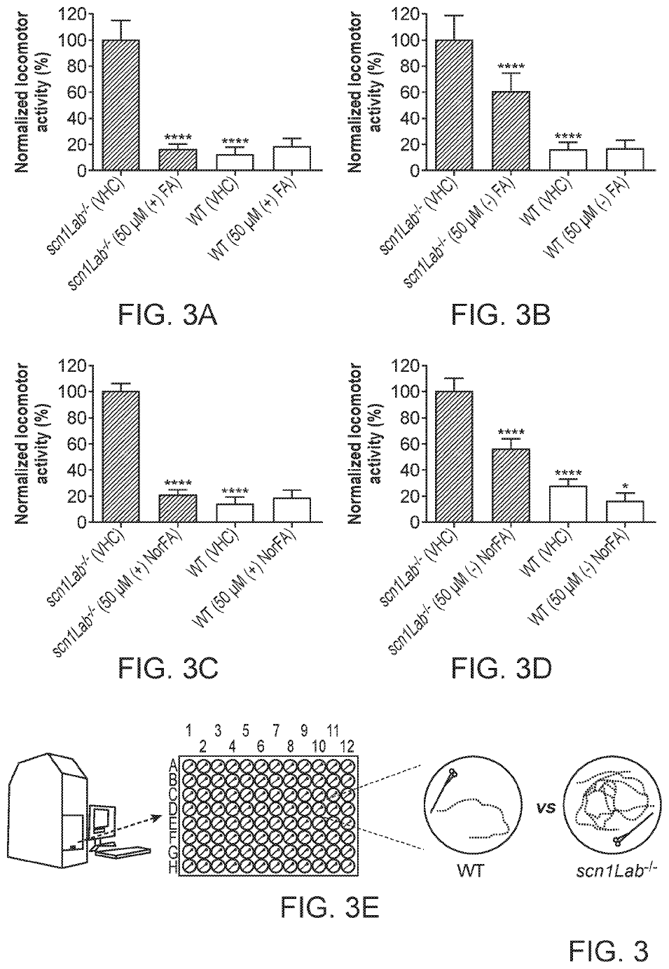

The present invention is based on several findings from studies of the fenfluramine enantiomers and norfenfluramine enantiomers in animals. The results demonstrate that dexfenfluramine is approximately twice as potent in treating symptoms of epilepsy compared to levofenfluramine. In the zebrafish locomotor assay, both enantiomers of fenfluramine showed antiseizure activity, suggesting that both contribute to the activity of (±)-fenfluramine. The efficacy profiles of (+)- and (−)-fenfluramine in comparison to that previously tested for (±)-fenfluramine suggest an additive effect of the enantiomers. (+)-fenfluramine is more efficacious than (−)-fenfluramine. FIGS. 3A and 3B demonstrate an 84% (dex) vs. 41% (levo) reduction in seizure behavior. The enantiomers of norfenfluramine also demonstrate antiseizure activity, suggesting that their activity also contributes to the activity of (±)-fenfluramine. In line with the findings for fenfluramine, (+)-norfenfluramine is more efficacious than (−)-norfenfluramine, i.e. 80% (dex) vs. 45% (levo) reduction in seizure behavior.

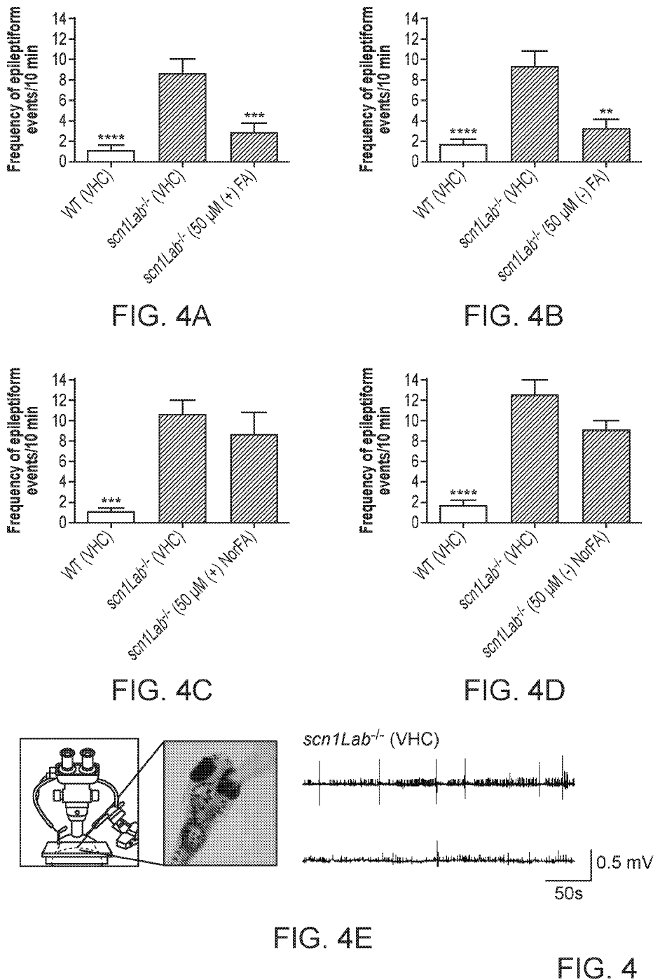

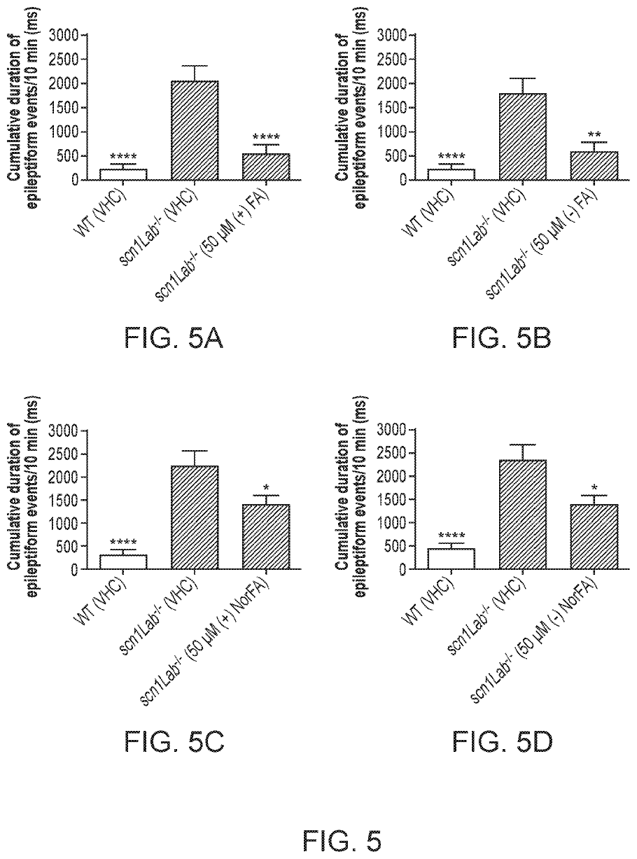

In line with the locomotor results, both enantiomers of fenfluramine demonstrated anti-epileptiform activity both in frequency of epileptiform events over 10 minutes of measurement and cumulative duration of epileptiform events recorded over 10 minutes, suggesting that both contribute to that of (±)-fenfluramine. The efficacy of (+)- and (−)-fenfluramine at the dose tested (50 micromolar) is comparable. The enantiomers of norfenfluramine also demonstrate anti-epileptiform activity, suggesting that their activity contributes to that of (±)-fenfluramine. However, (+)- and (−)-norfenfluramine are less efficacious than (+)- and (−)-fenfluramine. The efficacy of (+)- and (−)-norfenfluramine is comparable. See FIGS. 4 and 5.

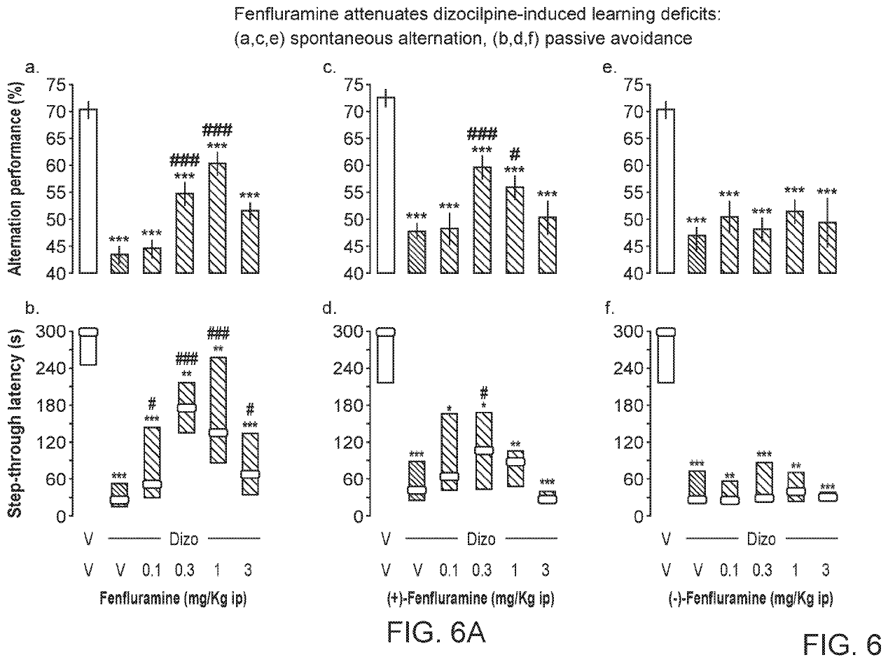

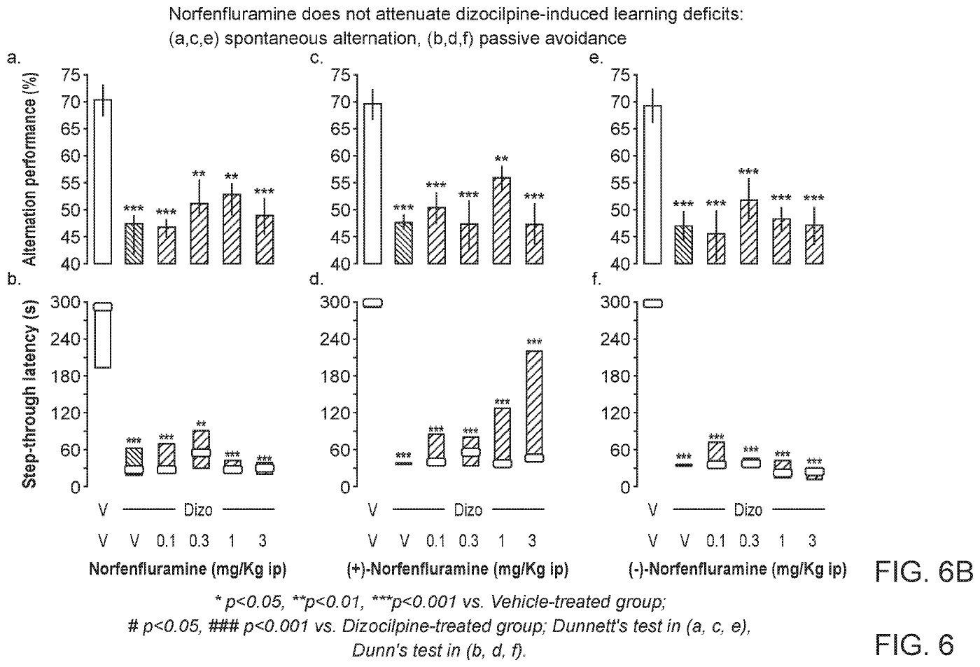

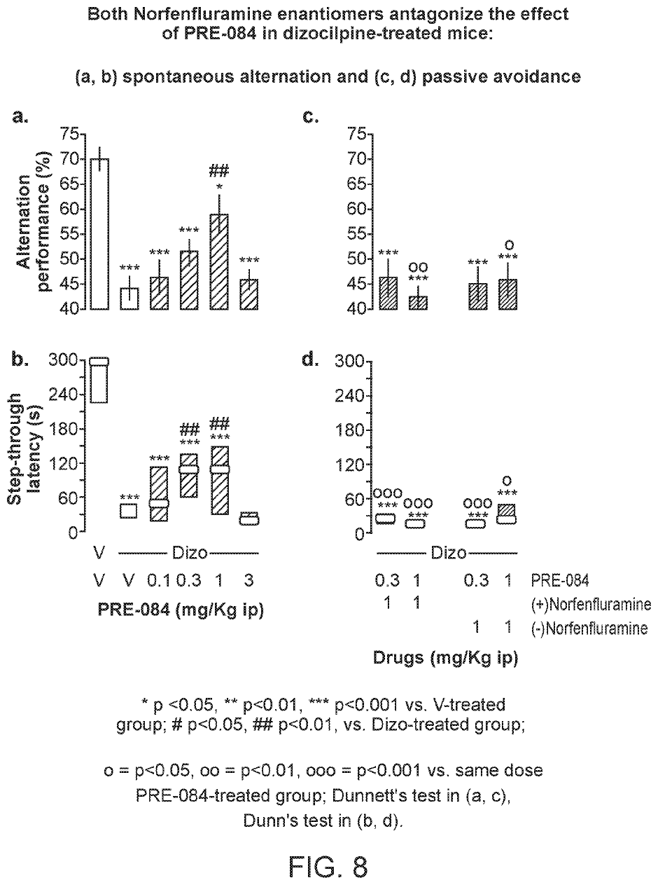

Studies of fenfluramine and norfenfluramine enantiomers activity as positive allosteric modulators of the sigma-1 receptor (S1R), alone or in conjunction with the known sigma-1 agonist, PRE-084, have provided mouse data in a dizocilpine-induced model of memory and associated learning deficits. (+)-Fenfluramine, but not (−)-FFA, attenuated dizocilpine-induced deficits in a similar manner as PRE-084 (FIG. 6A). The norfenfluramine racemate and its enantiomers did not affect dizocilpine amnesia (FIG. 6B) but prevented the effect of PRE-084 (FIG. 8) behaving similarly to S1R antagonists.

The combination of low doses of FFA or (+)-FFA, and PRE-084, followed by calculation of the combination index showed that lower dose combinations led to synergistic effects (Table 2 and Table 3). Calculations of CI for (−)-fenfluramine on the other hand showed an additive effect in the passive avoidance assay and an antagonistic effect in the Y-maze assay. These data therefore confirmed that FFA and its active isomer (+)-FFA behaved in vivo as S1R positive modulators. Both norfenfluramine enantiomers antagonized the effect of PRE-084 in both dizocilpine-treated mouse models. (See FIG. 8)

Therefore, in accordance with the invention, the disclosure provides methods for treating a patient diagnosed with a disease or disorder, including but not limited to a refractory epilepsy syndrome, such as Dravet syndrome, Lennox-Gastaut syndrome, Doose syndrome, Rett syndrome, West syndrome, Infantile Spasms and refractory seizures, by administering to the patient a therapeutically effective amount of a therapeutic agent consisting essentially of a single fenfluramine enantiomer. In one embodiment, the single fenfluramine enantiomer is dexfenfluramine. In one embodiment, the single fenfluramine enantiomer is dexfenfluramine. In alternate embodiments, the therapeutic agent can be administered as a monotherapy, as an adjunctive treatment to one or more antiepileptic drugs, in combination with one or more co-therapeutic agents, or with one or more agents which improve safety and/or efficacy relative to that observed when the therapeutic agent is used in the absence of such agents. Pharmaceutical compositions and formulations useful in practicing the methods disclosed and claimed herein are also provided. This invention provides the benefits of improving therapeutic efficacy and/or reducing cardiotoxicity relative to the efficacy and safety observed for racemic fenfluramine.

### Specific Aspects of the Invention

In one aspect, in accordance with the present invention, the disclosure provides methods of treating a disease or disorder by administering a therapeutic amount of dexfenfluramine to a patient. Also provided are methods of preventing, treating, or ameliorating symptoms associated with a disease or disorder in a patient diagnosed with the disease or disorder by administering a therapeutic amount of dexfenfluramine.

Diseases or disorders for which the methods disclosed herein find use include but not limited to patients diagnosed with refractory epilepsy, including but not limited to Dravet syndrome, Lennox-Gastaut syndrome, Doose syndrome, Rett syndrome, West syndrome, Infantile Spasms, and other refractory epilepsies. Symptoms for which the methods described herein are useful include but are not limited to seizures and seizure-induced respiratory arrest (S-IRA) leading to sudden unexpected death in epilepsy (SUDEP).

In one aspect, in accordance with the present invention, the disclosure provides pharmaceutical compositions and formulations that are useful in practicing the methods of the invention.

In another aspect, the disclosure provides methods of ameliorating memory and learning impairments associated with epileptic encephalopathies by administering dexfenfluramine or racemic fenfluramine to a patient. In an embodiment, dexfenfluramine is administered as the sole S1R modulating agent. In another embodiment, dexfenfluramine or racemic fenfluramine is co-administered with another S1R positive modulator, alone or in conjunction with the known sigma-1 agonist, PRE-084. In a further embodiment, the co-administration of dexfenfluramine or racemic fenfluramine with a S1R positive modulator and/or agonist provides synergistic effects to the patient.

In a further aspect, methods are provided of ameliorating memory and learning impairments associated with epileptic encephalopathies by administering to a patient dexfenfluramine or racemic fenfluramine to a patient in conjunction with another agent that inhibits CYP enzyme metabolism of fenfluramine to norfenfluramine. In an embodiment, the dexfenfluramine or racemic fenfluramine is co-administered with stiripentol. In another embodiment, the dexfenfluramine or racemic fenfluramine is co-administered with cannabidiol.

In yet another aspect, methods are provided of enhancing sigma-1 activity in a patient in need thereof by administering dexfenfluramine or racemic fenfluramine alone or in combination with a sigma-1 agonist. In an embodiment the dexfenfluramine or racemic fenfluramine in combination with the sigma-1 agonist provides synergistic effects in enhancing sigma-1 activity. In some embodiments, the sigma-1 agonist is chosen from the group consisting of PRE-084, fluvoxamine, ifenprodil, donepezil, sertraline, avanex 2-73, L-687,3834, dextromethorphan, amitriptyline, and neurosteroids, including dehydroepiandeterone (DHEA).

### Methods of Use

Single fenfluramine enantiomers can be employed in a variety of methods. As summarized above, aspects of the methods in accordance with the invention and disclosed herein include administering a therapeutically effective amount of a therapeutic agent consisting essentially of dexfenfluramine to treat a patient in need of treatment, for example, to a patient diagnosed with a disease or condition of interest, or to prevent, reduce or ameliorate symptoms of a disease or disorder in patients diagnosed with that disease or disorder.

Aspects of the method include administering a therapeutically effective amount of a therapeutic agent consisting essentially of dexfenfluramine to treat a patient in need of treatment, for example, to a patient diagnosed with a disease or condition of interest, or to prevent, reduce or ameliorate symptoms of a disease or disorder in patients diagnosed with that disease or disorder.

### Diseases and Disorders

As provided by the disclosure, therapeutic agents consisting essentially of a single fenfluramine enantiomer, whether administered alone, as an adjunctive treatment or in combination with a co-therapeutic agent, are useful in treating certain diseases and disorders, and/or in reducing, preventing or ameliorating their symptoms. In various embodiments of those methods, the single fenfluramine enantiomer is levofenfluramine. In various embodiments of those methods, the single fenfluramine enantiomer is dexfenfluramine.

Diseases and conditions of interest include, but are not limited to, seizure disorders such as epilepsy, particularly intractable forms of epilepsy, including but not limited to Dravet syndrome, Lennox-Gastaut syndrome, Doose syndrome, Rett syndrome, West syndrome, Infantile Spasms and refractory seizures, as well as other neurological related diseases, obesity, and obesity-related diseases. Also of interest is the prevention or amelioration of symptoms and co-morbidities associated with those diseases.

Single fenfluramine enantiomers also find use in preventing, adjunctively treating or ameliorating certain symptoms associated with those disorders, including seizures, particularly status epilepticus, seizure-induced respiratory arrest (S-IRA), and Sudden Unexplained Death in Epilepsy (SUDEP). In various embodiments of those methods, the single fenfluramine enantiomer is levofenfluramine. In various embodiments of those methods, the single fenfluramine enantiomer is dexfenfluramine.

### Monotherapy, Adjunctive Therapy and Co-therapies

In one aspect, in accordance with the present invention the disclosure provides methods of treatment wherein a therapeutic agent consisting essentially of a single fenfluramine enantiomer is administered as a monotherapy, or is used as an adjunctive therapy in combination with one or more antiepileptic agents, or is co-administered with one or more co-therapeutic agents or is co-administered with one or more metabolic inhibitors, including for example, stiripentol and cannabidiol. In some cases, the one or more agents being co-administered with the therapeutic agent having more than one activity.

In various embodiments of those methods, the single fenfluramine enantiomer is dexfenfluramine. In various embodiments of those methods, the single fenfluramine enantiomer is levofenfluramine.

In one embodiment, the therapeutic agent is provided as an adjunctive therapy in combination with one or more anti-epileptic agents.

Anti-epileptic agents of interest for use with the therapeutic agents disclosed herein include but are not limited to Acetazolamide, Carbamazepine, (Tegretol), Onfi (Clobazam), Clonazepam (Klonopin), Lamotrigine, Nitrazepam, Piracetam, Phenytoin, Retigabine, Stiripentol, Topiramate, and Carbatrol, Epitol, Equetro, Gabitril (tiagabine), Keppra (levetiracetam), Lamictal (lamotrigine), Lyrica (pregabalin), Gralise, Horizant, Neurontin, Gabarone (gabapentin), Dilantin, Prompt, Di-Phen, Epanutin, Phenytek (phenytoin), Topamax, Qudexy XR, Trokendi XR, Topiragen (topiramate), Trileptal, Oxtellar (oxcarbazepine), Depacon, Depakene, Depakote, Stavzor (valproate, valproic acid), Zonegran (zonisamide), Fycompa (perampanel), Aptiom (eslicarbazepine acetate), Vimpat (lacosamide), Sabril (vigabatrin), Banzel, Inovelon (rufinamide), Cerebyx (fosphenytoin), Zarontin (ethosuximide), Solfoton, Luminal (phenobarbital), Valium, Diastat (diazepam), Ativan (lorazepam), Lonopin, Klonopin (clonazepam), Frisium, Potiga (ezogabine), Felbatol (felbamate), Mysoline (primidone).

In some embodiments, the therapeutic agent is administered in combination with one or more agents which increase therapeutic efficacy, and provide additional therapeutic effects or improve safety, such as by reducing patient exposure to harmful metabolites, including but not limited to norfenfluramine. Of interest in this regard are agents which are metabolic inhibitors, as well as agents which have therapeutic effects themselves in addition to their effects on fenfluramine metabolism. Especially useful are cannabidiol and stiripentol. In addition to having antiseizure effects, they are also metabolic inhibitors which act on CYP450 enzymes that metabolize fenfluramine. When co-administered with fenfluramine, they increase fenfluramine blood plasma levels while simultaneously decreasing norfenfluramine levels, which results in reduced patient exposure to norfenfluramine for a given dose of fenfluramine, and which allows a reduced dose of fenfluramine to be administered to achieve the same therapeutic effect as a higher dose administered in the absence of those agents. The amount by which exposure to the single fenfluramine enantiomer increases and the amount by which norfenfluramine exposure decreases depends on factors including but not limited to the particular type and amount of co-therapeutic agent or agents with which the single enantiomer is being administered.

In some embodiments, the therapeutic agent is administered with a co-therapeutic agent capable of treating comorbid conditions associated with refractory epilepsy syndromes. Such conditions include but are not limited to psychiatric disorders (including but not limited to mood disorders such as depression and anxiety), cognitive disorders, migraine, and sleep disorders, cardiovascular disorders, respiratory disorders, inflammatory disorders, and other disorders, and sudden unexpected death in epilepsy (SUDEP), particularly in people with poorly controlled seizures. In various embodiments of those methods, the single fenfluramine enantiomer is dexfenfluramine. In various embodiments of those methods, the fenfluramine is administered as the racemate in combination with other agents. In some embodiments of the methods, dexfenfluramine is administered in combination with other agents.

### Genetic Testing

In some cases, it can be desirable to test the patients for a genetic mutation prior to administration of the therapeutic agents provided by the disclosure, especially in cases where use of specific agent is contraindicated either because the agent is ineffective or because it would have undesired or serious side effects. Thus, it is in some cases desirable to test patients prior to treatment. In the case of patients having Dravet syndrome, testing can be carried out for mutations in the SCN1A (such as partial or total deletion mutations, truncating mutations and/or missense mutations e.g. in the voltage or pore regions S4 to S6), SCN1 B (such as the region encoding the sodium channel β1 subunit), SCN2A, SCN3A, SCN9A, GABRG2 (such as the region encoding the γ2 subunit), GABRD (such as the region encoding the a subunit) and I or PCDH19 genes have been linked to Dravet syndrome.

Similarly, several reports in the literature evidence a strong, likely multifactorial genetic component for Doose syndrome (see e.g., Kelly et al., Developmental Medicine & Child Neurology 2010, 52: 988-993), and a number of mutations appear in a significant number of Doose syndrome patients, including sodium channel neuronal type 1 alpha subunit (SCN1A) mutations, sodium channel subunit beta-1 (SCN1B) and gamma-aminobutyric acid receptor, subunit gamma-2 (GABRG2) mutations; point mutations in exon 20 of SCN1A

In some instances, mutations in the methyl-CpG-binding protein 2 (MeCP2) gene on chromosome Xq28 cause Rett syndrome. Mutations in JMJD1C can also contribute to the development of the syndrome and intellectual disability (Genet Med. 2016 April; 18(4): 378-385). MeCP2 is highly expressed in the brain and is especially abundant in post-mitotic neurons. Most mutations are sporadic and rarely inherited. Moreover, mutations in males are frequently lethal in utero to hemizygous males or result in severe infantile encephalopathy because of complete absence of functional MeCP2. In contrast, females are heterozygous for the mutation with approximately one-half of the cells expressing the mutant MECP2 allele but with the other half expressing a functional allele, because of X chromosome inactivation. Thus, RTT is a disease that is almost exclusively seen in females. Approximately 95% of individuals with a Rett diagnosis have a confirmed mutation in MECP2. Hundreds of mutations in MECP2 have been identified, from which eight hotspot mutations account for more than 60% of all cases. Seizures and mental retardation are prominent features of this syndrome.

In some instances, the mutations occur in genes that are linked diseases and conditions characterized by various seizure types including, for example, generalized seizures, myoclonic seizures, absence seizures, and febrile seizures. Mutations can occur in one or more of the following genes: ALDH7A1, CACNA1A, CACNA1H, CACNB4, CASR, CHD2, CHRNA2, CHRNA4, CHRNB2, CLCN2, CNTN2, CSTB, DEPDC5, EFHC1, EPM2A, GABRA1, GABRB3, GABRD, GABRG2, GOSR2, GPR98, GRIN1, GRIN2A, GRIN2B, KCNMA1, KCNQ2, KCNQ3, KCTD7, MBD5, ME2, NHLRC1, PCDH19, PRICKLE1, PRICKLE2, PRRT2, SCARB2, SCN1A, SCN1B, SCN2A, SCN4A, SCN9A, SLC2A1, TBC1D24.

In some instances, the mutations occur in genes that are linked to age-related epileptic encephalopathies including, for example, early infantile epileptic encephalopathy. Mutations can occur in one or more of the following genes: ALDH7A1, ARHGEF9, ARX, CDKL5, CNTNAP2, FH, FOXG1, GABRG2, GRIN2A, GRIN2B, KCNT1, MAGI2, MAPK10, MECP2, NRXN1, PCDH19, PLCB1, PNKP, PNPO, PRRT2, RNASEH2A, RNASEH2B, RNASEH2C, SAMHD1, SCN1A, SCN1B, SCN2A, SCN8A, SCN9A, SLC25A22, SLC2A1, SLC9A6, SPTAN1, STXBP1, TCF4, TREX1, UBE3A, ZEB2.

In some instances, the mutations occur in genes that are linked to malformation disorders including, for example, neuronal migration disorders, severe microcephaly, pontocerebellar hypoplasia, Joubert syndrome and related disorders, holoprosencephaly, and disorders of the RAS/MAPK pathway. Mutations can occur in one or more of the following genes: AHI1, ARFGEF2, ARL13B, ARX, ASPM, ATR, BRAF, C12orf57, CASK, CBL, CC2D2A, CDK5RAP2, CDON, CENPJ, CEP152, CEP290, COL18A1, COL4A1, CPT2, DCX, EMX2, EOMES, FGF8, FGFR3, FKRP, FKTN, FLNA, GLI2, GLI3, GPR56, HRAS, INPP5E, KAT6B, KRAS, LAMA2, LARGE, MAP2K1, MAP2K2, MCPH1, MED17, NF1, NPHP1, NRAS, OFD1, PAFAH1B1, PAX6, PCNT, PEX7, PNKP, POMGNT1, POMT1, POMT2, PQBP1, PTCH1, PTPN11, RAB3GAP1, RAF1, RARS2, RELN, RPGRIP1L, SHH, SHOC2, SIX3, SLC25A19, SNAP29, SOS1, SPRED1, SRD5A3, SRPX2, STIL, TGIF1, TMEM216, TMEM67, TSEN2, TSEN34, TSEN54, TUBA1A, TUBA8, TUBB2B, VDAC1, WDR62, VRK1, ZIC2.

In some instances, the mutations occur in genes that are linked to epilepsy in X-linked intellectual disability. Mutations can occur in one or more of the following genes: ARHGEF9, ARX, ATP6AP2, ATP7A, ATRX, CASK, CDKL5, CUL4B, DCX, FGD1, GPC3, GRIA3, HSD17B10, IQSEC2, KDM5C, MAGT1, MECP2, OFD1, OPHN1, PAK3, PCDH19, PHF6, PLP1, PQBP1, RAB39B, SLC16A2, SLC9A6, SMC1A, SMS, SRPX2, SYN1, SYP.

In some instances, the mutations occur in genes that are linked to storage diseases and conditions characterized by organelle dysfunction including, for example, neuronal ceroid lipofuscinosis, lysosomal storage disorders, congenital disorders of glycosylation, disorders of peroxisome biogenesis, and leukodystrophies. Mutations can occur in one or more of the following genes: AGA, ALG1, ALG12, ALG2, ALG3, ALG6, ALG8, ALG9, ALG11, ALG13, ARSA, ARSB, ASPA, B4GALT1, CLN3, CLN5, CLN6, CLN8, COG1, COG4, COG5, COG6, COG7, COG8, CTSA, CTSD, DDOST, DOLK, DPAGT1, DPM1, DPM3, EIF2B1, EIF2B2, EIF2B3, EIF2B4, EIF2B5, FUCA1, GALC, GALNS, GFAP, GLB1, GNE, GNPTAB, GNPTG, GNS, GUSB, HEXA, HEXB, HGSNAT, HYAL1, IDS, IDUA, MCOLN1, MFSD8, MGAT2, MLC1, MOGS, MPDU1, MPI, NAGLU, NEU1, NOTCH3, NPC1, NPC2, PEX1, PEX12, PEX14, PEX2, PEX26, PEX3, PEX5, PEX6, PEX7, PEX10, PEX13, PEX16, PEX19, PGM1, PLP1, PMM2, PPT1, PSAP, RFT1, RNASEH2A, RNASEH2B, RNASEH2C, SAMHD1, SDHA, SGSH, SLC17A5, SLC35A1, SLC35A2, SLC35C1, SMPD1, SUMF1, TMEM165, TPP1, TREX1

In some instances, the mutations occur in genes that are linked to syndromic disorders with epilepsy including, for example, juvenile myoclonic epilepsy, childhood absence epilepsy, benign rolandic epilepsy, Lennox-Gastaut syndrome, Dravet syndrome, Ohtahara syndrome, West syndrome, Infantile Spasms, etc. Mutations can occur in one or more of the following genes: ATP2A2, ATP6V0A2, BCKDK, CACNA1A, CACNB4, CCDC88C, DYRK1A, HERC2, KCNA1, KCNJ10, KIAA1279, KMT2D, LBR, LGI1, MAPK10, MECP2, MEF2C, NDE1, NIPBL, PANK2, PIGV, PLA2G6, RAIL RBFOX1, SCN8A, SERPINI1, SETBP1, SLC1A3, SLC4A10, SMC3, SYNGAP1, TBX1, TSC1, TSC2, TUSC3, UBE3A, VPS13A, VPS13B

In some instances, the mutations occur in genes that are linked to the occurrence of migraines. Mutations can occur in one or more of the following genes: ATP1A2, CACNA1A, NOTCH3, POLG, SCN1A, SLC2A1.

In some instances, the mutations occur in genes that are linked to Hyperekplexia. Mutations can occur in the following genes: ARHGEF9, GLRA1, GLRB, GPHN, SLC6A5.

In some instances, the mutations occur in genes that are linked to inborn errors of metabolism including, for example, disorders of carbohydrate metabolism, amino acid metabolism disorders, urea cycle disorders, disorders of organic acid metabolism, disorders of fatty acid oxidation and mitochondrial metabolism, disorders of porphyrin metabolism, disorders of purine or pyridine metabolism, disorders of steroid metabolism, disorders of mitochondrial function, disorders of peroxisomal function, and lysosomal storage disorders. Mutations can occur in one or more of the following genes: ABAT, ABCC8, ACOX1, ACY1, ADCK3, ADSL, ALDH4A1, ALDH5A1, ALDH7A1, AMT, ARG1, ATIC, ATP5A1, ATP7A, ATPAF2, BCS1L, BTD, C120RF65, CABC1, COQ2, COQ9, COX10, COX15, DDC, DHCR7, DLD, DPYD, ETFA, ETFB, ETFDH, FOLR1, GAMT, GATM, GCDH, GCSH, GLDC, GLUD1, GLUL, HPD, HSD17B10, HSD17B4, KCNJ11, L2HGDH, LRPPRC, MGME1, MMACHC, MOCS1, MOCS2, MTHFR, MTR, MTRR, NDUFA1, NDUFA2, NDUFAF6, NDUFS1, NDUFS3, NDUFS4, NDUFS7, NDUFS8, NDUFV1, PC, PDHA1, PDHX, PDSS1, PDSS2, PGK1, PHGDH, POLG, PRODH, PSAT1, QDPR, RARS2, SCO2, SDHA, SLC19A3, SLC25A15, SLC46A1, SLC6A8, SUCLA2, SUOX, SURF1, TACO1, TMEM70, VDAC1.

Other genetic tests can be carried out, and can be required as a condition of treatment.

### Dosing

The therapeutic agents of the present invention can be dosed to patients in different amounts depending on different patient age, size, sex, condition as well as the particular use of the enantiomer.

For example, the dosing can be a daily dosing based on weight. However, for convenience the dosing amounts can be preset. In general, the smallest dose effective in a particular patient should be used. The patient can be dosed on a daily basis using a single dosage unit which single dosage unit can be comprised of a single fenfluramine enantiomer, for example levofenfluramine or dexfenfluramine, in an amount appropriate for the particular agent. The dosage unit can be selected based on the delivery route, e.g. the dosage unit can be specific for oral delivery, transdermal delivery, rectal delivery, buccal delivery, intranasal delivery, pulmonary delivery or delivery by injection.

Thus in some cases, a daily dose of less than about 10 mg/kg/day, such as less than about 10 mg/kg/day, less than about 9 mg/kg/day, less than about 8 mg/kg/day, less than about 7 mg/kg/day, less than about 6 mg/kg/day, less than about 5 mg/kg/day, less than about 4 mg/kg/day, less than about 3.0 mg/kg/day, less than about 2.5 mg/kg/day, less than about 2.0 mg/kg/day, less than about 1.5 mg/kg/day, less than about 1.0 mg/kg/day, such as about 1.0 mg/kg/day, about 0.95 mg/kg/day, about 0.9 meg/kg/day, about 0.85 mg/kg/day, about 0.8 mg/kg/day, about 0.75 mg/kg/day, about 0.7 mg/kg/day, about 0.65 mg/kg/day, about 0.6 mg/kg/day, about 0.55 mg/kg/day, about 0.5 mg/kg/day, about 0.45 mg/kg/day, about 0.4 mg/kg/day, about 0.350 mg/kg/day, about 0.3 mg/kg/day, about 0.25 mg/kg/day, about 0.2 mg/kg/day, about 0.15 mg/kg/day to about 0.1 mg/kg/day, about 0.075 mg/kg/day, about 0.05 mg/kg/day, about 0.025 mg/kg/day, about 0.0225 mg/kg/day, about 0.02 mg/kg/day, about 0.0175 mg/kg/day, about 0.015 mg/kg/day, about 0.0125 mg/kg/day, or about 0.01 mg/kg/day is employed.

Put differently, a preferred dose is less than about 10 to about 0.01 mg/kg/day. In some cases the dose is less than about 10.0 mg/kg/day to about 0.01 mg/kg/day, such as less than about 5.0 mg/kg/day to about 0.01 mg/kg/day, less than about 4.5 mg/kg/day to about 0.01 mg/kg/day, less than about 4.0 mg/kg/day to about 0.01 mg/kg/day, less than about 3.5 mg/kg/day to about 0.01 mg/kg/day, less than about 3.0 mg/kg/day to about 0.01 mg/kg/day, less than about 2.5 mg/kg/day to about 0.01 mg/kg/day, less than about 2.0 mg/kg/day to about 0.01 mg/kg/day, less than about 1.5 mg/kg/day to about 0.01 mg/kg/day, or less than about 1.0 mg/kg/day to 0.01 mg/kg/day, such as less than about 0.9 mg/kg/day, less than about 0.8 mg/kg/day, less than about less than about 0.7 mg/kg/day, less than about 0.6 mg/kg/day to about 0.01 mg/kg/day, less than about 0.5 mg/kg/day to about 0.01 mg/kg/day, less than about 0.4 mg/kg/day to about 0.01 mg/kg/day, less than about 0.3 mg/kg/day to about 0.01 mg/kg/day, or less than about.0.2 mg/kg/day to about 0.01 mg/kg/day. In some embodiments, the therapeutically effective dose of (+)-fenfluramine is from about 0.1 mg/kg/day to about 0.8 mg/kg/day.

As indicated above the dosing is based on the weight of the patient. However, for convenience the dosing amounts can be preset such as in the amount of 1.0 mg, 2.5 mg, 5 mg, 10 mg, 15 mg, 20 mg, 30 mg, 40 mg, or 50 mg. In certain instances, the dosing amount can be preset such as in the amount of about 0.25 mg to about 5 mg, such as about 0.25 mg, about 0.5 mg, about 0.75 mg, about 1.0 mg, about 1.25 mg, about 1.5 mg, about 1.75 mg, about 2.0 mg, about 2.25 mg, about 2.5 mg, about 2.75 mg, about 3.0 mg, about 3.25 mg, about 3.5 mg, about 3.75 mg, about 4.0 mg, about 4.25 mg, about 4.5 mg, about 4.75 mg, or about 5.0 mg.

In general, the smallest dose which is effective should be used for the particular patient.

The dosing amounts described herein can be administered one or more times daily to provide for a daily dosing amount, such as once daily, twice daily, three times daily, or four or more times daily, etc.

In certain embodiments, the dosing amount is a daily dose of 30 mg or less, such as 30 mg, about 29 mg, about 28 mg, about 27 mg, about 26 mg, about 25 mg, about 24 mg, about 23 mg, about 22 mg, about 21 mg, about 20 mg, about 19 mg, about 18 mg, about 17 mg, about 16 mg, about 15 mg, about 14 mg, about 13 mg, about 12 mg, about 11 mg, about 10 mg, about 9 mg, about 8 mg, about 7 mg, about 6 mg, about 5 mg, about 4 mg, about 3 mg, about 2 mg, or about 1 mg. In some cases, the dose is less than the dosing generally used in weight loss.

### Pharmaceutical Preparations

Also provided are pharmaceutical preparations. Pharmaceutical preparations are compositions that include a compound (either alone or in the presence of one or more additional active agents) present in a pharmaceutically acceptable vehicle. The term “pharmaceutically acceptable” means approved by a regulatory agency of the Federal or a state government or listed in the U.S. Pharmacopeia or other generally recognized pharmacopeia for use in mammals, such as humans. The term “vehicle” (sometimes abbreviated as “VHC,” “Veh” or “V” in figures) refers to a diluent, adjuvant, excipient, or carrier with which a compound of the invention is formulated for administration to a mammal.

Either of the fenfluramine enantiomers can be prepared and purified by skilled chemists using methods commonly known in the art. See e.g., Goument et al., Synthesis of (S)-fenfluramine from (R) or (S) 1-[3-(trifluoromethyl)phenyl]propan-2-ol, Bull. Soc. Chim Fr. (1993) 130, 450-458.

The pharmaceutical preparation can consist essentially of an optically pure fenfluramine enantiomer, such as essentially optically pure, such as, for example, an enantiomeric excess of 95% or more of dexfenfluramine. In alternate embodiments, the pharmaceutical preparation can include both fenfluramine enantiomers wherein the amount of the first fenfluramine enantiomer is different from the amount of the contaminating enantiomer, with the second “contaminating” enantiomer being present in an amount that is insufficient to have any or significant biological effects, such that any observable biological activity or therapeutic effect is attributable either entirely or substantially to the first fenfluramine enantiomer.

The amount of contaminating enantiomer that can be present in such compositions will vary according to the indication and/or symptom which is being treated.

For example, the pharmaceutical preparation can comprise both fenfluramine enantiomers wherein the amount of the first enantiomer is about 99.9%, about 99.8%, about 99.7%, about 99.6%, about 99.5%, about 99.4%, about 99.3%, about 99.4%, about 99.3%, about 99.2%, about 99.1%, about 99.0%, about 98%, about 97%, about 96%, about 95%, about 94%, about 93%, about 92%, about 91%, about 90%, about 85%, about 80%, about 75%, about 70%, about 65%, about 60%, about 55% or about 50% by weight of the total amount of fenfluramine present. Put another way, the first enantiomer is present in a range from about 99.9% to about 90%.

In general, the choice of excipient will be determined in part by the particular therapeutic agent, as well as by the particular method used to administer the composition. Accordingly, there is a wide variety of suitable formulations of the pharmaceutical composition of the present invention.

By way of illustration, the therapeutic agent can be admixed with conventional pharmaceutically acceptable carriers and excipients (i.e., vehicles) and used in the form of aqueous solutions, tablets, capsules, elixirs, suspensions, syrups, wafers, and the like. Such pharmaceutical compositions contain, in certain embodiments, from about 0.1% to about 90% by weight of the active compound, and more generally from about 1% to about 30% by weight of the active compound. The pharmaceutical compositions can contain common carriers and excipients, such as solubilizers, isotonic agents, suspending agents, emulsifying agents, stabilizers, preservatives, colorants, diluents, buffering agents, surfactants, moistening agents, flavoring agents and disintegrators, and including, but not limited to, corn starch, gelatin, lactose, dextrose, sucrose, microcrystalline cellulose, kaolin, mannitol, dicalcium phosphate, sodium chloride, alginic acid, vegetable or other similar oils, synthetic aliphatic acid glycerides, esters of higher aliphatic acids or propylene glycol, corn starch, potato starch, acacia, tragacanth, gelatin, glycerin, sorbitol, ethanol, polyethylene glycol, colloidal silicon dioxide, croscarmellose sodium, talc, magnesium stearate and stearic acid. Disintegrators commonly used in the formulations of this invention include croscarmellose, microcrystalline cellulose, corn starch, sodium starch glycolate and alginic acid. The compounds can be formulated into preparations for injection by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

### Routes of Administration

Administration of the therapeutic agent can be systemic or local. In certain embodiments, administration to a mammal will result in systemic release of the active compound (for example, into the bloodstream). Methods of administration can include enteral routes, such as oral, buccal, sublingual, and rectal; topical administration, such as transdermal and intradermal; and parenteral administration. Suitable parenteral routes include injection via a hypodermic needle or catheter, for example, intravenous, intramuscular, subcutaneous, intradermal, intraperitoneal, intraarterial, intraventricular, intrathecal, and intracameral injection and non-injection routes, such as intravaginal rectal, or nasal administration. In certain embodiments, the subject compounds and compositions are administered orally. In certain embodiments, it can be desirable to administer a compound locally to the area in need of treatment. In some embodiments, the method of administration of the subject compound is parenteral administration. This can be achieved, for example, by local infusion during surgery, topical application, e.g., in conjunction with a wound dressing after surgery, by injection, by means of a catheter, by means of a suppository, or by means of an implant, said implant being of a porous, non-porous, or gelatinous material, including membranes, such as sialastic membranes, or fibers.

### Dosage Forms

The dose of the therapeutic agent being administered in the methods of the present invention can be formulated in any pharmaceutically acceptable dosage form including, but not limited to oral dosage forms such as tablets including orally disintegrating tablets, capsules, lozenges, oral solutions or syrups, oral emulsions, oral gels, oral films, buccal liquids, powder e.g. for suspension, and the like; injectable dosage forms; transdermal dosage forms such as transdermal patches, ointments, creams; inhaled dosage forms; and/or nasally, rectally, vaginally administered dosage forms. Such dosage forms can be formulated for once a day administration, or for multiple daily administrations (e.g. 2, 3 or 4 times a day administration).

Dosage forms employed in the methods of the present invention can be prepared by combining it with one or more pharmaceutically acceptable diluents, carriers, adjuvants, and the like in a manner known to those skilled in the art of pharmaceutical formulation.

### Formulations

Particular formulations of the invention are in a liquid form. The liquid can be a solution or suspension and can be an oral solution or syrup which is included in a bottle with a pipette which is graduated in terms of milligram amounts which will be obtained in a given volume of solution. The liquid solution makes it possible to adjust the solution for small children which can be administered in increments appropriate to therapeutic agent, being administered.

In some embodiments, formulations suitable for oral administration can include (a) liquid solutions, such as an effective amount of the compound dissolved in diluents, such as water, or saline; (b) capsules, sachets or tablets, each containing a predetermined amount of the active ingredient, as solids or granules; (c) suspensions in an appropriate liquid; and (d) suitable emulsions. Tablet forms can include one or more of lactose, mannitol, corn starch, potato starch, microcrystalline cellulose, acacia, gelatin, colloidal silicon dioxide, croscarmellose sodium, talc, magnesium stearate, stearic acid, and other excipients, colorants, diluents, buffering agents, moistening agents, preservatives, flavoring agents, and therapeutically compatible excipients. Lozenge forms can include the active ingredient in a flavor, usually sucrose and acacia or tragacanth, as well as pastilles including the active ingredient in an inert base, such as gelatin and glycerin, or sucrose and acacia, emulsions, gels, and the like containing, in addition to the active ingredient, such excipients as are described herein.

In some cases, the therapeutic agent is formulated for oral administration. In some cases, for an oral pharmaceutical formulation, suitable excipients include pharmaceutical grades of carriers such as mannitol, lactose, glucose, sucrose, starch, cellulose, gelatin, magnesium stearate, sodium saccharine, and/or magnesium carbonate. For use in oral liquid formulations, the composition can be prepared as a solution, suspension, emulsion, or syrup, being supplied either in solid or liquid form suitable for hydration in an aqueous carrier, such as, for example, aqueous saline, aqueous dextrose, glycerol, or ethanol, preferably water or normal saline. If desired, the composition can also contain minor amounts of non-toxic auxiliary substances such as wetting agents, emulsifying agents, or buffers.

Particular formulations of the invention are in a liquid form. The liquid can be a solution or suspension and can be an oral solution or syrup which is included in a bottle with a pipette which is graduated in terms of milligram amounts which will be obtained in a given volume of solution. The liquid solution makes it possible to adjust the solution for small children which can be administered anywhere from 0.5 mL to 15 mL and any amount between in half milligram increments and thus administered in 0.5, 1.0, 1.5, 2.0 mL, etc.

A liquid composition will generally consist of a suspension or solution of the therapeutic agent or a pharmaceutically acceptable salt thereof in a suitable liquid carrier(s), for example, ethanol, glycerine, sorbitol, non-aqueous solvent such as polyethylene glycol, oils or water, with a suspending agent, preservative, surfactant, wetting agent, flavoring or coloring agent. Alternatively, a liquid formulation can be prepared from a powder for reconstitution.

### EXPERIMENTAL EXAMPLES

### Example 1

### Anti-Seizure Activity of Fenfluramine Enantiomers in SCN1a Mutant Zebrafish

Antiseizure activity of (−)-fenfluramine, (+)-fenfluramine, (−)-norfenfluramine and (+)-norfenfluramine in an SCN1a−/− mutant zebrafish model of Dravet syndrome were assessed and the results compared. For further detail on generation and use of the zebrafish model see, for example, Zhang Y, et al. (2015) PLoS ONE 10(5): e0125898, doi:10.1371/journal.pone.0125898.

Zebrafish embryos (Danio rerio) heterozygous for the scn1Lab mutation (scn1Lab+/−) are backcrossed with Tupfel longfin wildtype (WT scn1Lab+/+) were used in measuring anti-epileptic activities substantially as reported by Sourbron, J. et al, ACS Chem. Neurosci., 2016, 7 (5), pp 588-598.

The point mutation in heterozygous or homozygous scn1Lab mutants makes it possible to distinguish them from WT scn1Lab^{+/+} by genotyping. In heterozygous scn1Lab^{+/-} mutants the PCR product contains AT3632G (wildtype allele) and AG3632G (allele with point mutation). The point mutation converts a thymine (AT3632G) into a guanine (AG3632G), which transforms a methionine (M) to an arginine (R). Digestion with PagI results in two fragments of different length (250 and 500 base pairs). The PCR product of adult WT scn1Lab^{+/+} zebrafish, on the contrary, only contains AT3632G and hence, after PagI digestion, only one fragment will be visible (250 base pairs). Homozygous scn1Lab^{-/-} mutants solely have AG3632G. As PagI only recognizes AT3632G, genotyping of these homozygous mutants results in one visible fragment (500 base pairs). Moreover, sequencing data (LGC Genomics) confirmed the genetic difference of heterozygous scn1Lab^{+/-} mutants (T-G mutation) compared to wildtype scn1Lab^{+/+}.

As compared to WT larvae, homozygous scn1Lab^{-/-} mutants exhibit an increased locomotor activity expressed as total distance in large movements (lardist), a surrogate marker for seizure behavior. (Baraban et al, 2013). Additional phenotypes of these mutants include: abnormal optokinetic response (OKR; 5 dpf); darkened pigmentation; death by 14 dpf; spontaneous seizure-like activity; abnormal (forebrain electrographic activity (3-7 dpf); seizure activity; lower levels of serotonin in scn1lab^{-/-} head homogenates at 7 dpf; zebrafish express orthologs of human serotonin (5HT) receptor subtypes at 5 dpf; nighttime hyperactivity (5 dpf); in open field-increased thigmotaxis. decreased movement (at 5 dpf); no differences in GABAergic neurons compared to wild type (5 dpf); 5-HT2a (hitr2aa/b) and S-HT2c; (htr2c11) orthologs expressed in larval heads in wild-type and scn1lab−/− mutants and in adult wild-type brains.

Adult zebrafish are housed at 28.0° C., on a 14/10 hour light/dark cycle under standard aquaculture conditions. Fertilized eggs are collected via natural spawning. Anaesthetized fish (tricaine 0.02%) are fin-clipped and genotyped by PCR. After genotyping, samples are purified (MinElute PCR Purification Kit) and sequenced by LGC Genomics. Age-matched Tupfel longfin wildtype larvae are used as control group (WT scn1Lab+/+). These embryos and larvae are kept on a 14/10 hour light/dark cycle in embryo medium (Danieaus): 1.5 mM HEPES, pH 7.6, 17.4 mM NaCl, 0.21 mM KCl, 0.12 mM MgSO4, and 0.18 mM Ca(NO3)2 in an incubator at 28.0° C. All zebrafish experiments carried out were approved by the Ethics Committee of the University of Leuven (Ethische Commissie van de KU Leuven, approval number (061/2013) and by the Belgian Federal Department of Public Health, Food Safety & Environment (Federale Overheidsdienst Volksgezondheid, Veiligheid van de Voedselketen en Leefmileu, approval number LA1210199).

To assay the locomotor activity of homozygous scn1Lab−/− mutants and control WT scn1Lab+/+, zebrafish larvae were placed one larva per well in a 96-well plate in 100 μL of embryo medium from 4 to 8 days post-fertilization (dpf). Each day the larvae are tracked in an automated tracking device (ZebraBox™ apparatus; Viewpoint, Lyon, France) for 10 min after 30 min habituation (100-second integration interval). All recordings were performed at the same time during daytime period. The total distance in large movements was recorded and quantified using ZebraLab™ software (Viewpoint, Lyon, France). Data was pooled together from at least three independent experiments with at least 24 larvae per condition.

Epileptiform electrical activity was assayed by open-field recordings in the zebrafish larval forebrain at 7 dpf. Homozygous scn1Lab−/− mutants and control WT scn1Lab+/+ were embedded in 2% low-melting-point agarose (Invitrogen) to position a glass electrode into the forebrain. This glass electrode was filled with artificial cerebrospinal fluid (aCSF) made from: 124 mM NaCl, 2 mM KCl, 2 mM MgSO4, 2 mM CaCl2, 1.25 mM KH2PO4, 26 mM NaHCO₃ and 10 mM glucose (resistance 1-5 MΩ) and connected to a high-impedance amplifier. Subsequently, recordings were performed in current clamp mode, low-pass filtered at 1 kHz, high-pass filtered 0.1 Hz, digital gain 10, at sampling intervals of 10 μs (MultiClamp 700B amplifier, Digidata 1440A digitizer, both Axon instruments, USA). Single recordings were performed for 10 min. Epileptiform activity was quantified according to the duration of spiking paroxysms as described previously (Orellana-Paucar et al, 2012). Electrograms were analyzed with the aid of Clampfit 10.2 software (Molecular Devices Corporation, USA). Spontaneous epileptiform events were taken into account when the amplitude exceeded three times the background noise and lasted longer than 50 milliseconds (ms). This threshold was chosen due to the less frequent observation of epileptiform events in wildtype ZF larvae with a shorter duration than 50 ms.

Racemic fenfluramine, dexfenfluramine and levofenfluramine (“test compounds:) were obtained from Peak International Products B.V. Functional analogs (agonists) and antagonists were chosen based on their high and selective affinity (except for ergotamine, see further) for the different 5-HTsubtype receptors (Ki in nanomolar range), and on their log P value (i.e. >1, expected to exhibit a good bioavailability in zebrafish larvae (Milan, 2003)). Compounds are obtained from Tocris Bioscience, except for 5-HT2A-antagonist (ketaserine), 5-HT4-agonist (cisapride) and 5-HT5A-agonist (ergotamine) that are purchased from Sigma-Aldrich. Compounds are dissolved in dimethyl sulfoxide (DMSO, 99.9% spectroscopy grade, Acros Organics) and diluted in embryo medium to achieve a final DMSO concentration of 0.1% w/v, 0.1% w/v DMSO in embryo medium also served as a vehicle control (VHC).

To evaluate the maximal tolerated concentration (MTC) of each compound, 6 dpf-old WT scn1Lab+^{+/+} zebrafish larvae were incubated in a 96-well plate (tissue culture plate, flat bottom, FALCON®, USA) with different concentrations of compound or VHC at 28° C. on a 14/10 hour light/dark cycle under standard aquaculture conditions (medium is replenished daily). Each larva was individually checked under the microscope during a period of 48 hours for the following signs of toxicity: decreased or no touch response upon a light touch of the tail, loss of posture, body deformation, edema, changes in heart rate or circulation and death. The maximum tolerated concentration (MTC) was defined as the highest concentration at which no signs of toxicity were observed in 12 out of 12 zebrafish larvae within 48 hours of exposure to sample. MTC was determined as 100 micromolar (μM) for (+)-fenfluramine and both norfenfluramine enantiomers. A MTC of 50 μM was determined for (−)-fenfluramine.

Scn1Lab^{-/-} mutants and WT scn1Lab+/+ larvae are arrayed in the same plate and treated at 6 days post fertilization (dpf) with the test compounds (25 μM), or VHC in individual wells of a 96-well plate. After incubation at 28° C. on a 14/10 hour light/dark cycle and 30-min chamber habituation 6 and 7 dpf larvae are tracked for locomotor activity for 10 min (100-second integration interval) under dark conditions. An incubation time of 1.5 hours is further referred to as short treatment (6 dpf). Furthermore, these larvae are analyzed after more than 22 hours incubation (7 dpf), i.e. long treatment. The total locomotor activity is quantified using the parameter lardist and plotted in cm. Data for each compound is pooled together from multiple independent experiments with at least 9 larvae per treatment condition.

Epileptiform activity is measured by open-field recordings in the zebrafish larval forebrain at 7 dpf, as described above. Scn1Lab−/− mutants and WT scn1Lab+/+ larvae are incubated with the test compounds (25 μM) or VHC alone, on 6 dpf for a minimum of 22 hours (long treatment). Recordings of 7 dpf larvae, from at least 8 scn1Lab−/− mutant larvae are taken per experimental condition. For treated WT scn1Lab+/+ larvae at least 5 per condition are analyzed, due to the scarce observation of epileptiform activity in wildtype larvae. Electrographic recordings are quantified for the different treatment conditions.

The heads of 7 dpf-old zebrafish larvae are used to determine the amount of the neurotransmitters dopamine, noradrenaline and serotonin present. Six heads per tube are homogenized on ice for one min in 100 μl 0.1 M antioxidant buffer (containing vitamin C). Homogenates are centrifuged at 15 000 g for 15 min at 4° C. Supernatants (70 μl) are transferred to a sterile tube and stored at −80° C. until analysis.

The neurotransmitter determination is based on the microbore LC-ECD method (Sophie Sarre, Katrien Thorré, Ilse Smolders, 1997) and done in collaboration with the Center for Neurosciences, C4N, VUB (Brussels, Belgium). The chromatographic system consisted of a FAMOS microautosampler of LC Packings/Dionex (Amsterdam, The Netherlands), a 307 piston pump of Gilson (Villiers-le-Bel, France), a DEGASYS DG-1210 degasser of Dionex and a DECADE II electrochemical detector equipped with a μ-VT03 flow cell (0.7 mm glassy carbon working electrode, Ag/AgCl reference electrode, 25 μm spacer) of Antec (Zoeterwoude, The Netherlands). The mobile phase is a mixture of 87% V/V aqueous buffer solution at pH 5.5 (100 mM sodium acetate trihydrate, 20 mM citric acid monohydrate, 2 mM sodium decanesulfonate, 0.5 mM disodium edetate) and 13% V/V acetonitrile. This mobile phase is injected at a flow rate of 60 μL/min. The temperature of the autosampler tray is set on 15° C. and the injection volume is 10 pt. A microbore UniJet C8 column (100×1.0 mm, 5 μm) of Bioanalytical Systems (West Lafayette, Ind., United States) is used as stationary phase. The separation and detection are performed at 35° C., with a detection potential of +450 mV vs Ag/AgCl. Data acquisition is carried out by Clarity chromatography software version 3.0.2 of Data Apex (Prague, The Czech Republic). The amount of neurotransmitter (in nmol) is calculated based on the total mass of six heads.

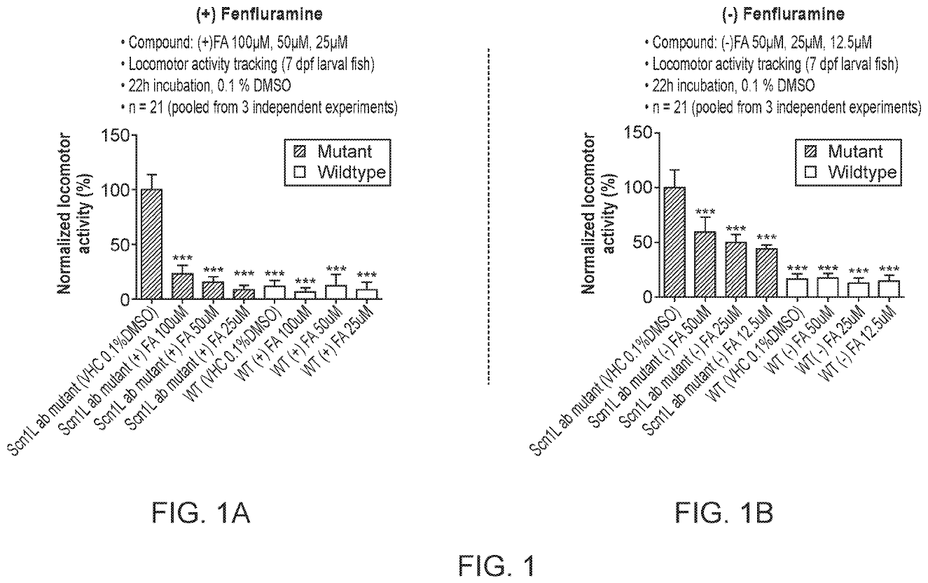

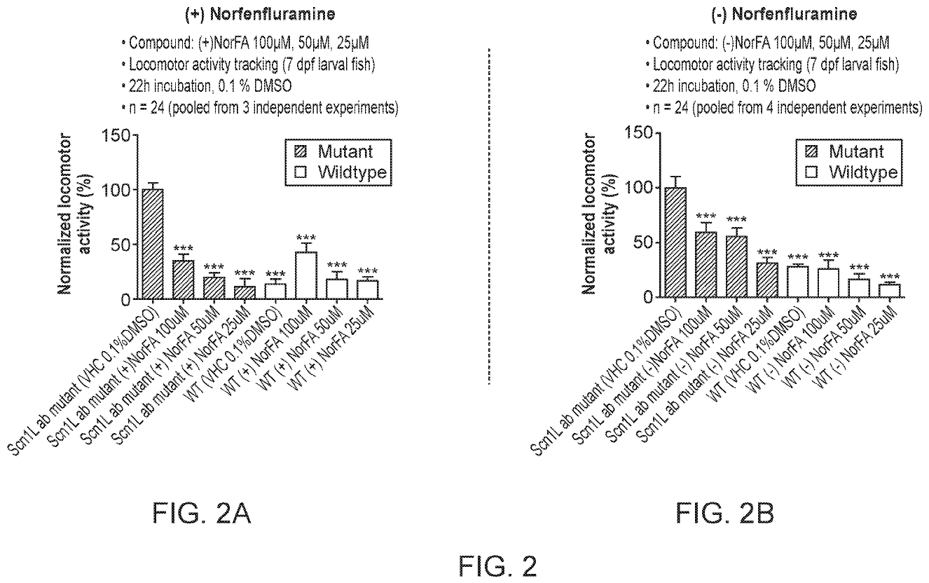

Statistical analyses are performed using GraphPad Prism 5 software (GraphPad Software, Inc.). The larval locomotor activity is evaluated by using One-way ANOVA, followed by Dunnett's multiple comparison tests. Values are presented as means±standard deviation (SD). LFP (local forebrain potential) measurements of electrographic brain activity were analyzed by a Mann-Whitney test. Statistically significant differences (p<0.05) between a treatment group and the equivalent control groups (scn1Lab−/− mutant or WT scn1Lab+/+) were considered indicative of a decrease or increase in locomotor or electrographic brain activity of zebrafish larvae. The neurotransmitter amount of scn1Lab−/− mutants is compared with WT scn1Lab+/+ larvae by a Student's t-test because all data passed the normality test (D'Agostino & Pearson omnibus normality test). Data collected from the studies are displayed in FIGS. 1 and 2. Analysis and interpretations are presented in Table 1 below.

TABLE 1

| | Dravet Zebrafish Scn1a^{-/-} mutants | |
|---|---|---|
| Doses and route of | 25, 59 and 100 μM; oral via embryo medium | |
| administration | | |
| | Locomotor Assay Behavior | LFP Assay |
| Fenfluramine | | |
| (+)-Fenfluramine | Dose-dependent decrease in | Most effective in reducing spikes |
| | locomotor behavior | from local area of brain |
| | Low dose more effective than | Normal dose-response; higher |
| | high dose | dose is more effective |
| (−)-Fenfluramine | Less effective than (+)- | Also reduces spikes but less |
| | fenfluramine | effective than the (+)- |
| | Dose-dependent decrease in | enantiomer |
| | locomotor behavior | Normal dose-response; higher |
| | Low dose more effective than | dose is more effective |
| | high dose | |
| Norfenfluramine | | |
| (+)-Norfenfluramine | Similar pattern as (+)-FFA | Less effective than (+)-FFA; |
| (−)-Norfenfluramine | Similar pattern as (−)-FFA | Inactive |

| | |
|---|---|
| Conclusions | (+)-FFA is more effective than racemic FFA |
| | Locomotor (low dose most effective) vs LFP (high dose most effective) |
| | Locomotor results shows effects of compound on motor neurons (system |
| | response) vs LFP showing result of compound on neurons touched by tip |
| | of electrode (localized firing) |

### Example 2

### Anti-Seizure Effects of Dexfenfluramine in 6 Hz Mouse Models

Rapid screening of possible anticonvulsants can be performed by using acute (instead of chronic) animal models of drug-resistant seizures. One example is the acute 6-Hz model, (part of the Epilepsy Therapy Screening Program (ETSP)) which is useful as a drug screening platform for drug-resistant seizures when the intensity is set on 44 mA (Leclercq et al., 2014) and 32 mA (Wilcox et al., 2013). This idea is based on the fact that 44 mA 6-Hz seizures are resistant to several AEDs and even sodium valproate and levetiracetam are less potent at this relatively higher intensity (44 compared to 22 mA). Furthermore, this model can detect compounds with novel modes of action since it does not fully discriminate compounds based on their mechanism of action (Barton et al., 2001). In addition to the protocol using 44 mA pulses, more “lenient” versions of the model using 22 mA or 32 mA currents are sometimes employed.

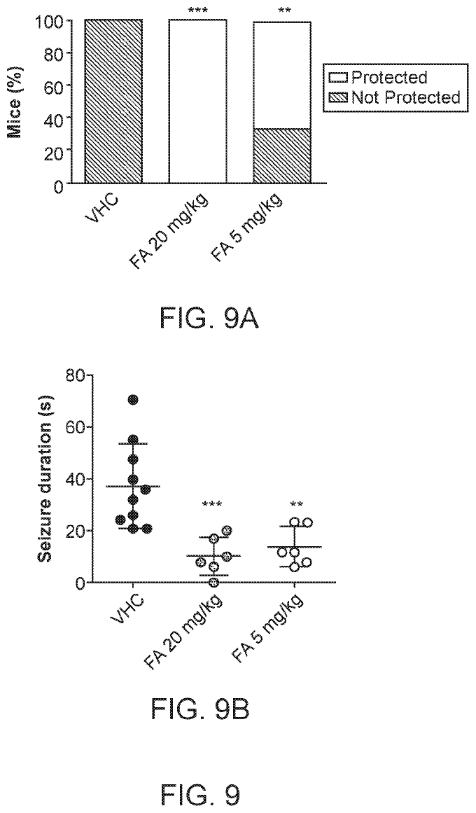

Results are shown in FIGS. 9A and 9B. Racemic fenfluramine significantly reduced seizures in the mouse 6-Hz model (Mann-Whitney test; p<0.05 vs. VHC). A dose-dependent decrease in number of mice having seizures and in duration of seizures was observed. Additionally, the mice injected with vehicle displayed a period of post-seizure aggression, whereas the mice treated with fenfluramine did not. Thus, racemic fenfluramine is effective in reducing seizures in an animal model of refractory epilepsy.

### 2(a) Anti-Seizure Effects of Racemic Fenfluramine in a Mes, 6 Hz/44MA and Corneal Kindled Mouse Models

The antiseizure effects of racemic fenfluramine were assessed in three mouse models of acute and chronic seizures in naïve male CF-1 mice (Envigo (Indianapolis, Ind., USA); 18-35 g): 1) the acute maximal electroshock (MES) test; 2) the acute 44 mA 6 Hz test; and 3) the chronic corneal kindled mouse (CKM) model.

Methods: Testing progressed in three phases: identification, time course, and dose response. In the identification phase, mice were administered FFA (3, 10, 30 mg/kg, ip), vehicle, or retigabine (positive control, 20 or 50 mg/kg, ip) 0.5 or 2 hr before testing (n=4/group). The time course of antiseizure activity of the most effective dose without minimal motor impairment (MMI) was determined at 0.25, 0.5, 1, 2, and 4 hr after dosing (n=4/time point). Dose-dependent anticonvulsant activity of FFA was assessed by testing between 0.25 and 120 mg/kg (n=8/dose). The effect of FFA on mild motor impairment (MMI) in the fixed-speed rotarod (FSR) was assessed prior to the MES, 44 mA 6 Hz, and CKM tests. The doses at which 50% of mice were protected from seizures (ED50) or exhibited MMI (TD50) and 95% CI's were estimated by Probit regression.

### 2(B) Anti-Seizure Effects of Fenfluramine Enantiomers in a Mouse Model

| Mouse Model | Type of Model | Comparison to Other AEDs |
|---|---|---|
| Maximal | Generalized tonic-clonic seizures | Sodium channel blocking |
| Electroshock | Provides indication of ability of the compound to | agents such as carbamazepine, |
| | prevent seizure spread when all neuronal circuits in the | phenytoin and lamotrigine |
| | brain are maximally active | show activity in this assay |
| 6 Hz 44 mA | Models secondarily generalized focal seizures | Retigabine, tiagabine, clobazam |
| | | exhibit activity in this mode |
| Corneal | Pharmacological profile consistent with hippocampal | Identifies compounds with use |
| Kindled | kindled rat | similar to levetiracetam |
| | Consistent with human partial epilepsy | |

| Model | Identification and Time Course |
|---|---|
| MES | Identification - using racemic fenfluramine |
| | 0.5 hours - 0%, 0% and 100% protection at doses of 3, 10 and 30 mg/kg |
| | 2.0 hours - 75%, 50% and 100% protection at doses of 3, 10 and 30 mg/kg |
| | Time Course at last time point (4-hrs post dosing) |
| | 100% protection at 4 hours post dosing |
| | ED50 at: 2.9 mg/kg (95% CI 1.4 to 5.1 mg/kg) |
| | Impairment |
| | No impairment on rotarod at doses used in this model |
| | Test to be repeated with (+)-fenfluramine and (−)-fenfluramine |
| 6 HZ 44 mA | Identification - using racemic fenfluramine |
| | Time Course - at 0.5 hrs post dosing: |
| | Peak activity |
| | ED50: 47.0 mg/kg (95% CI 31.9 to 66.7 mg/kg) |
| | TD5034.6 mg/kg (95% CI 25.3 to 53.0) |
| | Impairment |
| | Some impairment found at a dose of 30 mg/kg at timepoints of 0.25, 0.50, 1.00, and |
| | 2.00 hours; no impairment and no protection at 4 hours |
| | Test to be repeated with (+)-fenfluramine and (−)-fenfluramine |
| Corneal | Identification --using racemic fenfluramine |
| kindled | Minimal anticonvulsant activity at all doses tested |
| | Impairment |
| | Some mice died at doses of 90 mg/kg (2 of 8) and 120 mg/kg (6 of 8) doses |
| | Test to be repeated with (+)-fenfluramine and (−)-fenfluramine |

### 2(B) Conclusion

Acute administration of racemic fenfluramine exerted substantial anticonvulsant effect in the MES model, an observation that aligns with the clinical experience of treating these seizures in Dravet patients. Racemic fenfluramine shows some activity in a model of focal seizures that secondarily generalize. Data from tests of fenfluramine and norfenfluramine stereoisomers in these models can be compared to the activity of the racemate.

### Example 3

Experiments were conducted to determine the dose-response effects of fenfluramine (FEN) and norfenfluramine (NOR), racemate and (+)- and (−)-isomers, in an in vivo mouse model; the dizocilpine-induced amnesia model (described below) is used to test responses to drugs acting at the sigma-1 receptor (S1R) (Maurice et al., 1994a,b; Maurice, et al., 1998, Neuroscience 83:413-428).

### Materials:

Animals: Male Swiss OF-1 mice, aged 7-9 weeks and weighing 32±2 g were purchased from Janvier (St Berthevin, France). Mouse housing and experiments took place within the animal facility of the University of Montpellier (CECEMA, registration number D34-172-23). Animals were housed in groups with access to food and water ad libitum. They were kept in a temperature and humidity-controlled facility on a 12 h/12 h light/dark cycle (lights on at 7:00 h). Behavioral experiments were carried out between 9:00 h and 17:00 h, in a sound-attenuated and air-regulated experimental room, to which mice were habituated for 30 min. All animal procedures were conducted in strict adherence to the European Union Directive of Sep. 22, 2010 (2010/63).

### Drugs and Injections:

2-(4-Morpholinethyl)-1-phenylcyclohexanecarboxylate hydrochloride (PRE-084) and (5S,10R)-(+)-5-Methyl-10,11-dihydro-5H-dibenzo [a,d] cyclohepten-5,10-imine hydrogen maleate ((+)-MK-801, dizocilpine) were from Sigma-Aldrich (Saint-Quentin-Fallavier, France).

4-Methoxy-3-(2-phenylethoxy)-N,N-dipropylbenzeneethanamine hydrochloride (NE-100) was from Tocris Bioscience (Bristol, UK). Drugs were solubilized in physiological saline. Steroids were solubilized in pure sesame oil (Sigma-Aldrich) (=vehicle solutions). They were administered intraperitoneally (IP), for drugs, or subcutaneously (SC), for steroids, in a volume of 100 μl per 20 g body weight.

Statistical Analyses

Data were analyzed using a one-way analysis of variance (ANOVA, F value), followed by a Dunnett's test or a Kruskal-Wallis non-parametric ANOVA (H value), followed by a Dunn's multiple comparison tests, for passive avoidance latencies (expressed as median and interquartile range). The level of statistical significance wasp<0.05.

Calculations of Combination Index

Isobologram analyses evaluating the nature of the interaction between two drugs at a given effect level were performed according to Fraser's concept (1872). (See also Zhao, L., et al., 2010, Front. Biosci. 2:241-249; Maurice, et al., 2016, Behav. Brain. Res., 296:270-278). The concentrations required to produce a given effect (eg, IC₅₀) are determined for drug A (IC_{x,A}) and drug B (IC_{x,B}) and indicated on the x and y axes of a two-coordinate plot, forming the two points (IC_{x,A}, 0) and (0, IC_{x,B}). The line connecting these two points is the line of additivity. Then, the concentrations of A and B contained in the combination that provide the same effect, denoted as (C_{A,x}, C_{B,x}), are placed in the same plot. Synergy, additivity, or antagonism are indicated when (C_{A,x}, C_{B,x}) is located below, on, or above the line, respectively. Operationally, a combination index (CI) is calculated as:

CI=C_{A,x}/IC_{x,A}+C_{B,x}/IC_{x,B}

where C_{A,x} and C_{B,x} are the concentrations of drug A/B used in a combination that generates x % of the maximal combination effect; CI is the combination index; IC_{x,A/B} is the concentration of drug A/B needed to produce x % of the maximal effect. A CI of less than, equal to, or more than 1 indicates synergy, additivity, or antagonism, respectively. Calculations for CI for several combinations of PRE-084 and (i) racemic fenfluramine (Table 2); (ii) (+)-fenfluramine (Table 3); and (iii) (−)-fenfluramine (Table 4) were performed using data from the Y-maze assay (spontaneous alternation) and the Step-through/Passive avoidance assays.

Dizocilpine (MK-801, (1S,9R)-1-methyl-16-azatetracyclo[7.6.1.0^{2,7}.0^{10,15}] hexadeca2,4,6,10,12,14-hexaene) is an antagonist of the N-methyl-D-aspartate receptor in the glutamate category involved with the central nervous system and displays a variety of physiological actions, such as anesthetic and anticonvulsant properties. Dizocilpine also interferes with memory and long-term potentiation. Racemic fenfluramine has been demonstrated to be a positive allosteric modulator (PAM) of sigma-1 receptors (see US 20180092864, which is incorporated by reference for all purposes herein) was investigated by testing fenfluramine's ability to prevent the effects of dizocilpine's negative effects on memory in two complementary behavioral tests assessing short- and long-term memories, as described below and in Maurice, T, et al., Pharmacol Biochem Behav. 1994b, 49(4):859-69.

PRE-084 (2-(4-Morpholinyl)ethyl 1-phenylcyclohexanecarboxylate hydrochloride), a selective sigma 1 agonist, and racemic fenfluramine or dexfenfluramine were tested alone and in combination in Swiss mice in two assays: (i) the step-through passive avoidance assay and spontaneous alternation in a Y-maze assay. PRE-084 and (+)-norfenfluramine and (−)-norfenfluramine were also tested alone and in combination in Swiss mice. The drugs were administered intraperitoneally (“ip” or “IP”) with dizocilpine (0.15 mg/kg) and tested in the spontaneous alternation test on day 1 and in the passive avoidance test on days 2-3.

The two behavioral responses measured were (1) spontaneous alternation in the Y-maze (YMT, spatial working memory) and (2) passive avoidance (STPA, non-spatial long-term memory).

### Step-Through Passive Avoidance

The Step-through passive avoidance test assesses non-spatial/contextual long-term memory and was performed as previously described (Meunier et al., 2006, Br. J. Pharmacol. 149:998-1012; Maurice, et al., 2016, Behav. Brain. Res., 296:270-278). The apparatus consisted of a 2-compartment box, with one illuminated with white polyvinylchloride (PVC) walls and a transparent cover (15×20×15 cm high), one with black polyvinylchloride walls and cover (15×20×15 cm high), and a grid floor. A guillotine door separated each compartment. A 60 W lamp was positioned 40 cm above the apparatus lit the white compartment during the experimental period. Scrambled foot shocks (0.3 mA for 3 s) were delivered to the grid floor using a shock generator scrambler (Lafayette Instruments, Lafayette, Mass., USA). The guillotine door was initially closed during the training session. Each mouse was placed into the white compartment. After 5 s, the door was raised. When the mouse entered the darkened compartment and placed all its paws on the grid floor, the door was gently closed and the 3-scrambled foot shock was delivered for 3 s. The step-through latency, i.e., the latency spent to enter the dark compartment, and the level of sensitivity to the shock were recorded. The latter was evaluated as: 0=no sign; 1=flinching reactions; 2=flinching and vocalization reactions. The retention test was carried out 24 h after training. Each mouse was placed again into the white compartment. After 5 s, the door was raised. The step-through latency was recorded up to 300 s. Animals entered the darkened compartment or were gently pushed into it and the escape latency, i.e., the time spent to return into the white compartment, was also measured up to 300 s. Results were expressed as median and interquartile (25%-75%) range.

### Spontaneous Alternation in the Y Maze

Animals were tested for spontaneous alternation performance in the Y-maze, an index of spatial working memory (Maurice et al., 1994a and 1994b; Maurice, 1998, Neuroscience 83:413-428; Meunier et al., 2006, Br. J. Pharmacol. 149:998-1012; Maurice, et al., 2016, Behav. Brain. Res., 296:270-278). The Y-maze is made of grey PVC. Each arm is 40 cm long, 13 cm high, 3 cm wide at the bottom, 10 cm wide at the top, and converged at an equal angle. Each mouse was placed at the end of one arm and allowed to move freely through the maze during an 8-min session. The series of arm entries, including possible returns into the same arm, were checked visually. An alternation was defined as entries into all three arms on consecutive occasions. The number of maximum alternations was therefore the total number of arm entries minus two and the percentage of alternation was calculated as: actual alternations/maximum alternations)×100. Parameters included the percentage of alternation (memory index) and total number of arm entries (exploration index).

Results of the assays of the Y-maze assay (spontaneous alternation performance) and the Step-through/Passive avoidance assays with fenfluramine and its enantiomers (6A) and norfenfluramine and its enantiomers (6B) as the sole study drug are shown in FIG. 6.

As shown in FIG. 6A, the fenfluramine racemate (a,b) and FFA enantiomers ((+)-fenfluramine and ((−)-fenfluramine) were studied for their effects on dizocilpine-induced learning impairments in the Y-maze test and passive avoidance tests. Animals were injected intraperitonally with the fenfluramine racemate, (+)-fenfluramine, or (−)-fenfluramine (0.1-0.3 mg/kg) 10 min before they received dizocilpine (Dizo, 0.15 mg/kg ip), and 20 min before the Y-maze test session or the passive avoidance training session. Retention was tested after 24 h, without further drug treatment. Data from the vehicle-treated group are shown as 100% and the Dizo-treated group as 0%. The mean±SEM is shown in FIG. 6A (a, c, e) and median and interquartile range in FIG. 6A (b, d, f, with protection shown using a cursor-on-scale representation). ANOVA: F(5,78)=35.8, p<0.0001, n=12-19 in FIG. 6A(a); F(5,69)=15.7, p<0.0001, n=10-14 in FIG. 6A(c); F(5,68)=8.52, p<0.0001, n=11-12 in FIG. 6A(e). Kruskal-Wallis ANOVA: H=39.9, p<0.0001, n=12-17 in FIG. 6A(b); H=30.3, p<0.0001, n=11-13 in FIG. 6A(d); H=21.7, p<0.001, n=11-12 in FIG. 6A(f). * p<0.05, ** p<0.01, *** p<0.001 vs. V-treated group; #p<0.05, ###p<0.001 vs. Dizo-treated group; Dunnett's test in 6A (a, c, e), Dunn's test in 6A (b,d,f).

As shown in FIG. 6B, the Norfenfluramine racemate and the Norfenfluramine enantiomers ((+)-Norfenfluramine and (−)Norfenfluramine) were studied for their effects on dizocilpine-induced learning impairments in the Y-maze test and passive avoidance tests. Animals were injected intraperitonally with the Norfenfluramine racemate, (+)-Norfenfluramine, or (−)-Norfenfluramine (0.1-0.3 mg/kg) 10 min before they received dizocilpine (Dizo, 0.15 mg/kg ip), and 20 min before the Y-maze test session or the passive avoidance training session. Retention was tested after 24 h, without further drug treatment. The mean±SEM is shown in FIG. 6B (a, c, e) and median and interquartile range in FIG. 6B (b, d, f, with protection shown using a cursor-on-scale representation). ANOVA: F(5,67)=5.77, p<0.001, n=10-12 in FIG. 6B(a); F(5,64)=5.98, p<0.001, n=10-12 in FIG. 6B(c); F(5,58)=7.49, p<0.0001, n=9-10 in FIG. 6B(e). Kruskal-Wallis ANOVA: H=24.6, p<0.0001, n=10-12 in FIG. 6B(b); H=24. p<0.0001, n=10-11 in FIG. 6B(d); H=31.1, p<0.0001, n=9-11 in FIG. 6B(f). * p<0.05, ** p<0.01, *** p<0.001 vs. V-treated group; #p<0.05, ###p<0.001 vs. Dizo-treated group; Dunnett's test in 6B (a, c, e), Dunn's test in 6B (b, d, f).

Overall, this line of experimentation confirmed that the fenfluramine racemate (FFA) and its active isomer (+)-FFA significantly attenuated both deficits and the most active doses appeared to be 0.3 and 1 mg/kg IP for both drugs (FIG. 6A, a-d). The profiles for FFA racemate and its dextrogyre isomer are highly coherent as would be expected from a sigma-1 acting drug; in other words, (FFA) and (+)-FFA behaved in vivo as S1R positive modulators. In contrast, the (−) isomer (−)-FFA (FIG. 6A, e,f), the Norfenfluramine racemate, and the Norfenfluramine enantiomers ((+)-Norfenfluramine and (−)Norfenfluramine) were not active on dizocilpine-induced learning deficits in tests (FIGS. 6B a-f).

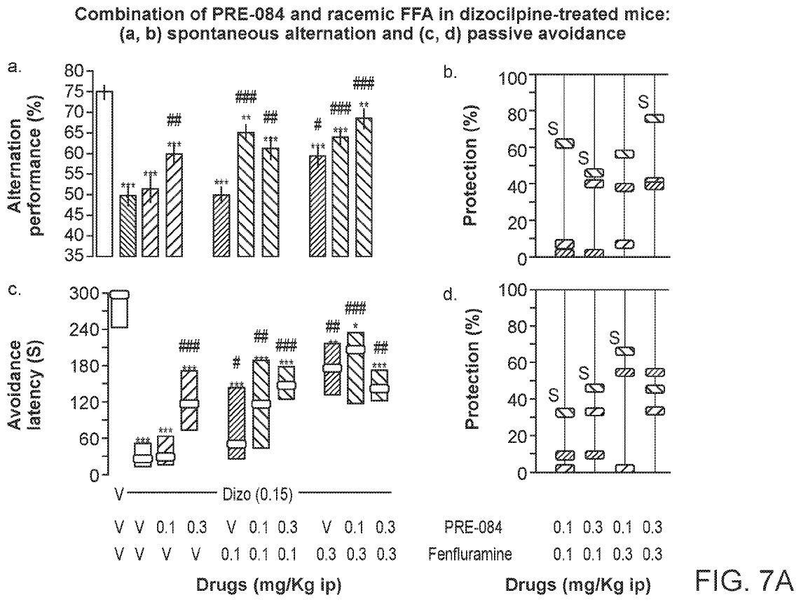

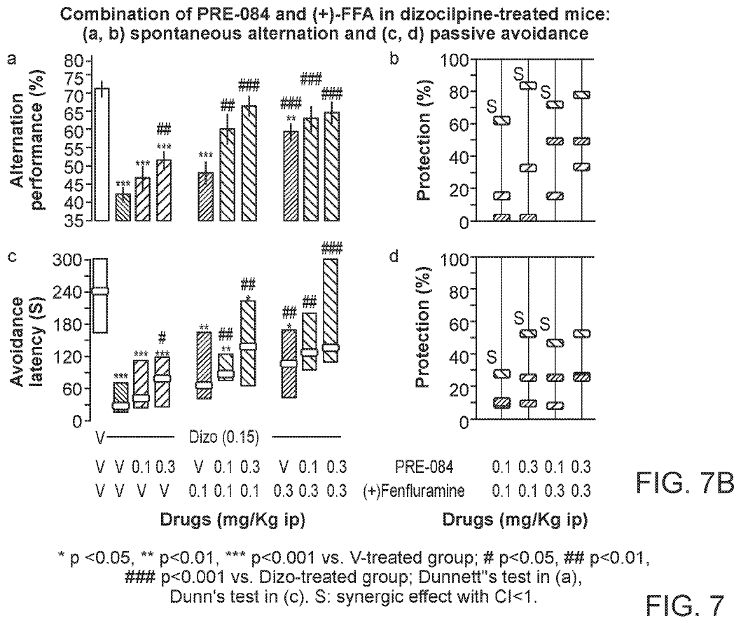

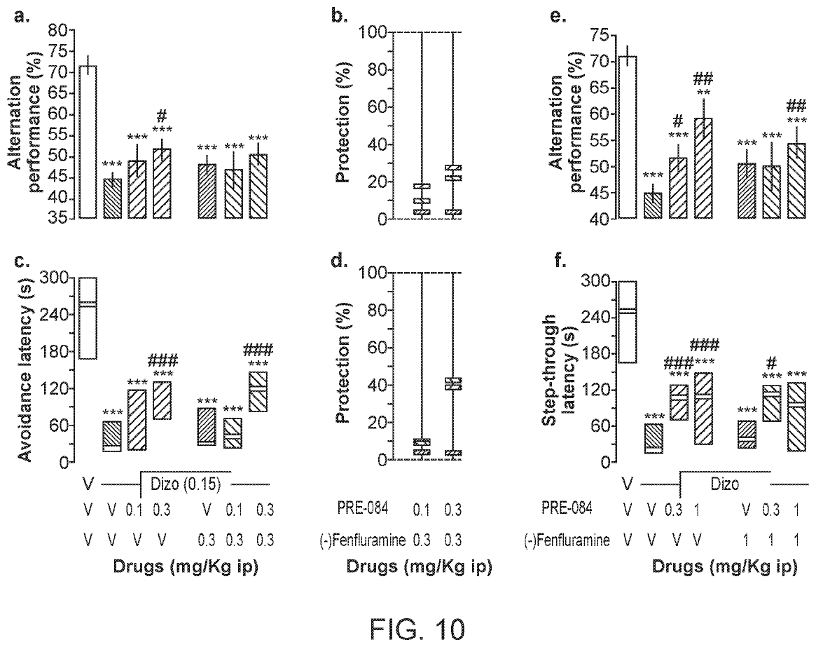

Combination studies between PRE-084 and racemic fenfluramine or (+)-fenfluramine in dizocilpine-treated mice are shown in FIGS. 7 and 10, and Tables 2 and 3. Assay conditions for the Y-maze assay (spontaneous alternation) and the Step-through/Passive avoidance assays with racemic fenfluramine (FIG. 7A) or (+)-fenfluramine (FIG. 7B) in combination with PRE-084 were as follows:

As shown in FIG. 7A, PRE-084 and/or fenfluramine racemate were studied for their effects on the dizocilpine-induced learning impairments. Animals were injected intraperitonally with PRE-084 and/or fenfluramine racemate (each at 0.1-0.3 mg/kg) 10 min before they received dizocilpine (Dizo, 0.15 mg/kg ip), and 20 min before the spontaneous alternation performance in the Y-maze (FIG. 7A, (a,b)) and step-through latency in the passive avoidance test (FIG. 7A, (c,d)). Retention was tested after 24 h, without further drug treatment. Data show mean±SEM in FIG. 7A (a) and median and interquartile range in FIG. 7A (c). In FIG. 7A (b,d), protection is shown using a cursor-on-scale representation, with data from the V-treated group as 100% and the Dizo-treated group as 0%. ANOVA: F_{(9,133)}=16.1, p<0.0001, n=11-20 per group, in FIG. 7A(a); H=58.1, p<0.0001, n=11-18 per group, in FIG. 7A(c). * p<0.05, ** p<0.01, *** p<0.001 vs. V-treated group; #p<0.05, ##p,<0.01, ###p<0.001 vs. Dizo-treated group; Dunnett's test in 7A (a), Dunn's test in 7A (c). S indicates synergistic effect with CI<1.

As shown in FIG. 7B, PRE-084 and/or (+)-fenfluramine were studied for their effects on the dizocilpine-induced learning impairments. Animals were injected intraperitonally with PRE-084 and/or fenfluramine racemate (each at 0.1-0.3 mg/kg) 10 min before they received dizocilpine (Dizo, 0.15 mg/kg ip), and 20 min before the spontaneous alternation performance in the Y-maze (FIG. 7B, (a,b)) and step-through latency in the passive avoidance test (FIG. 7B, (c,d)). Retention was tested after 24 h, without further drug treatment. Data show mean±SEM in FIG. 7B (a) and median and interquartile range in FIG. 7B (c). In FIG. 7B (b,d), protection is shown using a cursor-on-scale representation, with data from the V-treated group as 100% and the Dizo-treated group as 0%. ANOVA: F_{(9,123)}=11.7, p<0.0001, n=11-14 per group, in FIG. 7B (a); H=37.5, p<0.0001, n=12-14 per group, in FIG. 7B(c). * p<0.05, ** p<0.01, *** p<0.001 vs. V-treated group; #p<0.05, ##p,<0.01, ###p<0.001 vs. Dizo-treated group; Dunnett's test in FIG. 7B (a), Dunn's test in FIG. 7B (c). S indicates synergistic effect with CI<1.

The combination of low doses of FFA or (+)-FFA, and PRE-084, followed by calculation of the combination index showed that lower dose combinations led to synergistic effects (Table 2 and Table 3). These data therefore confirmed that FFA and its active isomer (+)-FFA behaved in vivo as S1R positive modulators.

FIG. 8 presents four graphs that show that both norfenfluramine enantiomers antagonize the effect of PRE-084 in dizocilpine-treated mice: (a, b) spontaneous alternation and (c, d) passive avoidance. Graphs show mean±SEM (vertical line) in (a) and median (black bar) and interquartile range (shaded bar) in (c).

FIG. 9 includes two graphs that summarize the effects of racemic fenfluramine (abbreviated herein as “FA,” “FFA” or “FEN”) treatment in 6-Hz mice, as described in Example 3A. FIG. 9A is a bar graph showing the percentage of animals protected for mice treated with vehicle, with 20 mg FA, and with 5 mg/kg FA. FIG. 9B shows the effects on seizure duration. **p<0.01 and ***p<0.001 vs. VHC-injected; n=6-10 NMRI mice for all experimental conditions. Protection being defined as the absence of a seizure within the expected time frame (i.e. below the minimum seizure duration in VHC treated mice).

FIG. 10(a-f): Calculations of CI for (−)-fenfluramine on the other hand showed an additive effect in the passive avoidance assay and an antagonistic effect in the Y-maze assay. Both norfenfluramine enantiomers antagonized the effect of PRE-084 in both dizocilpine-treated mouse models. FIG. 10 and Table 4: show studies of the combination of PRE-084 and (−)-fenfluramine on dizocilpine-induced learning deficits, as measured by spontaneous alternation performance in the Y-maze (a, b) and step-through latency in the passive avoidance test (c, d). PRE-084 (0.1-1 mg/kg ip) and/or (−)Fenfluramine (0.3, 1 mg/kg ip) were injected 10 min before dizocilpine (Dizo, 0.15 mg/kg intraperitoneally), 20 min before the Y-maze test session or the passive avoidance training session, retention being tested after 24 h, without further drug treatment. Data show mean±SEM in (a, e) and median and interquartile range in (c, f). In (b, d), protection is shown using a cursor-on-scale representation, with data from vehicle (V)-treated group as 100% and Dizo-treated group as 0% in (a, c). ANOVA: F(6,89)=11.2, p<0.0001, n=10-15 per group, in (a); H=47.2, p<0.0001, n=9-19 per group, in (c); F(4,66)=15.0, p<0.0001, n=10-15 in (e); H=40.3, p<0.0001, n=9-19 per group, in (f). * p<0.05, *** p<0.001 vs. V-treated group; #p<0.05, ##p<0.01, ###p<0.001 vs. Dizo-treated group; Dunnett's test in (a, e), Dunn's test in (c, f).

As observed in FIGS. 10 (a,c) (calculated as percentage of protection in FIGS. 10 (b,d)), (−)Fenfluramine was without any effect on PRE-084 efficacy. The drug was also tested at a higher dose (1 mg/kg) on higher, effective doses of PRE-084 (0.3-1 mg/kg) to determine whether the drug could behave as an antagonist. As shown in FIGS. 10 (e, f), (−) Fenfluramine was without effect on PRE-084 efficacy.

Therefore, the (−)-fenfluramine is not active against dizocilpine-induced learning deficits and does not behave as a S1R antagonist. (See Table 4).

TABLE 2
Calculation of combination index (CI) for the
PRE-084/racemic Fenfluramine coadministration.

| | | Cx, | Cx, | |
|---|---|---|---|---|
| Treatment (mg/kg IP) | PP (%) | PRE-084 | Fenfluramine | CI |
| (a) Y-Maze | | | | |
| PRE-084 (0) | 0.0 ± 9.0 | | | |
| PRE-084 (0.1) | 6.6 ± 12.8 | | | |
| PRE-084 (0.3) | 40.5 ± 9.2 ¹ | | | |
| Fenfluramine (0) | 0.0 ± 9.0 | | | |
| Fenfluramine (0.1) | 1.3 ± 7.9 | | | |
| Fenfluramine (0.3) | 37.9 ± 9.4 | | | |
| Fenfluramine (1) | 59.8 ± 6.1 ² | | | |
| PRE-084 (0.1) + Fenfluramine (0.1) | 61.5 ± 6.8 | 0.46 ± 0.05 | 0.96 ± 0.08 | 0.32 ± 0.03 |
| PRE-084 (0.1) + Fenfluramine (0.3) | 55.9 ± 6.1 | 0.42 ± 0.04 | 0.87 ± 0.07 | 0.58 ± 0.05 |
| PRE-084 (0.3) + Fenfluramine (0.1) | 45.6 ± 10.5 | 0.35 ± 0.04 | 0.70 ± 0.06 | 1.01 ± 0.09 |
| PRE-084 (0.3) + Fenfluramine (0.3) | 75.1 ± 9.5 | 0.56 ± 0.06 | 1.19 ± 0.10 | 0.79 ± 0.07 |
| (a) StepThrough/Passive Avoidance | | | | |
| PRE-084 (0) | 0.0 ± 3.8 | | | |
| PRE-084 (0.1) | 4.9 ± 9.9 | | | |
| PRE-084 (0.3) | 34.6 ± 10.5 ³ | | | |
| Fenfluramine (0) | 0.0 ± 3.8 | | | |
| Fenfluramine (0.1) | 11.0 ± 11.6 | | | |
| Fenfluramine (0.3) | 55.7 ± 10.5 | | | |
| Fenfluramine (1) | 40.8 ± 13.0 ⁴ | | | |
| PRE-084 (0.1) + Fenfluramine (0.1) | 33.7 ± 11.0 | 0.30 ± 0.03 | 0.55 ± 0.05 | 0.51 ± 0.06 |
| PRE-084 (0.1) + Fenfluramine (0.3) | 66.8 ± 12.9 | 0.58 ± 0.05 | 1.51 ± 0.12 | 0.58 ± 0.08 |
| PRE-084 (0.3) + Fenfluramine (0.1) | 46.3 ± 7.6 | 0.41 ± 0.04 | 0.91 ± 0.09 | 0.84 ± 0.09 |
| PRE-084 (0.3) + Fenfluramine (0.3) | 43.8 ± 10.5 | 0.39 ± 0.04 | 0.84 ± 0.07 | 1.13 ± 0.15 |

Percent protection (PP) was calculated using 100% for V-treated animals and 0% for Dizocilpine-treated animals. YMT: Y-maze test, STPA: step-through passive avoidance, CI: combination index.
C_{x, Drug} was calculated using the linear regression from responses with the drug alone:
¹ y = 139.89x − 2.938;
² y = 60.06x + 3.734;
³ y = 120.14x − 2.827;
⁴ y = 34.44x + 14.8.

TABLE 3
Calculation of combination index (CI) for the PRE-084/(+)-Fenfluramine coadministration.

| | | Cx, | Cx, | |
|---|---|---|---|---|
| Treatment (mg/kg IP) | PP (%) | PRE-084 | (+)Fenfluramine | CI |
| (a) Y-Maze | | | | |
| PRE-084 (0) | 0.0 ± 6.4 | | | |
| PRE-084 (0.1) | 15.4 ± 10.9 | | | |
| PRE-084 (0.3) | 32.6 ± 8.9 ¹ | | | |
| (+)Fenfluramine (0) | 0.0 ± 8.6 | | | |
| (+)Fenfluramine (0.1) | 1.3 ± 7.9 | | | |
| (+)Fenfluramine (0.3) | 37.9 ± 9.4 ² | | | |
| PRE-084 (0.1) + (+)Fenfluramine (0.1) | 61.0 ± 13.6 | 0.56 ± 0.05 | 0.39 ± 0.04 | 0.43 ± 0.04 |
| PRE-084 (0.1) + (+)Fenfluramine (0.3) | 71.7 ± 11.7 | 0.66 ± 0.06 | 0.45 ± 0.04 | 0.81 ± 0.07 |
| PRE-084 (0.3) + (+)Fenfluramine (0.1) | 82.8 ± 8.4 | 0.77 ± 0.07 | 0.52 ± 0.05 | 0.58 ± 0.05 |
| PRE-084 (0.3) + (+)Fenfluramine (0.3) | 77.1 ± 9.8 | 0.71 ±0.06 | 0.49 ± 0.05 | 1.04 ± 0.10 |
| (a) StepThrough/PassiveAvoidance | | | | |
| PRE-084 (0) | 0.0 ± 6.6 | | | |
| PRE-084 (0.1) | 7.6 ± 12.4 | | | |
| PRE-084 (0.3) | 25.2 ± 9.8 ³ | | | |
| (+)Fenfluramine (0) | 0.0 ± 6.7 | | | |
| (+)Fenfluramine (0.1) | 9.0 ± 15.8 | | | |
| (+)Fenfluramine (0.3) | 24.8 ± 14.9 ⁴ | | | |
| PRE-084 (0.1) + (+)Fenfluramine (0.1) | 27.3 ± 12.5 | 0.33 ± 0.03 | 0.33 ± 0.04 | 0.61 ± 0.07 |
| PRE-084 (0.1) + (+)Fenfluramine (0.3) | 46.1 ± 15.4 | 0.55 ± 0.05 | 0.56 + 0.07 | 0.73 ± 0.08 |
| PRE-084 (0.3) + (+)Fenfluramine (0.1) | 52.1 ± 16.1 | 0.62 ± 0.06 | 0.63 ± 0.08 | 0.64 ± 0.07 |
| PRE-084 (0.3) + (+)Fenfluramine (0.3) | 51.6 ± 16.1 | 0.61 ± 0.06 | 0.63 ± 0.08 | 0.97 ± 0.11 |

Percent protection (PP) was calculated using 100% for V-treated animals and 0% for Dizocilpine-treated animals. YMT: Y-maze test, STPA: step-through passive avoidance, CI: combination index.
C_{x, Drug} was calculated using the linear regression from responses with the drug alone:
¹ y = 105.36x + 1.9393;
² y = 171.06x − 5.9699;
³ y = 84.66x − 0.331;
⁴ y = 81.99x + 0.329.

TABLE 4
Calculation of combination index (CI) for the PRE-084/(−)Fenfluramine coadministration.

| | | Cx, | Cx, | |
|---|---|---|---|---|
| Treatment (mg/kg IP) | PP (%) | PRE-084 | (−)Fenfluramine | CI |
| (a) Y Maze | | | | |
| PRE-084 (0) | 0.0 ± 6.6 | | | |
| PRE-084 (0.1) | 17.2 ± 13.8 | | | |
| PRE-084 (0.3) | 27.0 ± 9.5 ¹ | | | |
| (−)Fenfluramine (0) | 0.0 ± 9.9 | | | |
| (−)Fenfluramine (0.3) | 3.2 ± 8.1 | | | |
| (−)Fenfluramine (1) | 13.8 ± 10.5 ² | | | |
| PRE-084 (0.1) + (−)Fenfluramine (0.3) | 9.3 ± 15.6 | 0.07 ± 0.01 | 0.69 ± 0.07 | 1.89 ± 0.18 |
| PRE-084 (0.3) + (−)Fenfluramine (0.3) | 22.3 ± 9.8 | 0.22 ± 0.02 | 1.62 ± 0.15 | 1.53 ± 0.15 |
| (a) StepThrough/PassiveAvoidance | | | | |
| PRE-084 (0) | 0.0 ± 4.6 | | | |
| PRE-084 (0.1) | 8.8 ± 11.0 | | | |
| PRE-084 (0.3) | 38.9 ± 10.4 ³ | | | |
| (−)Fenfluramine (0) | 0.0 ± 6.0 | | | |
| (−)Fenfluramine (0.3) | 3.9 ± 8.4 | | | |
| (−)Fenfluramine (1) | 7.3 ± 13.8 ⁴ | | | |
| PRE-084 (0.1) + (−)Fenfluramine (0.3) | 12.7 ± 8.1 | 0.11 ± 0.01 | 1.73 ± 0.16 | 1.09 ± 0.10 |
| PRE-084 (0.3) + (−)Fenfluramine (0.3) | 41.9 ± 11.8 | 0.33 ± 0.03 | 5.98 ± 0.56 | 0.96 ± 0.09 |

Percent protection (PP) was calculated using 100% for V-treated animals and 0% for Dizocilpine-treated animals. YMT: Y-maze test, STPA: step-through passive avoidance, CI: combination index.
C_{x, Drug} was calculated using the linear regression from responses with the drug alone:
¹ y = 84.25x + 3.5206;
² y = 14.02x − 0.417;
³ y = 6.89x + 0.743;
⁴ y = 132.7x − 1.78.

### Example 4

### Effects of Combining Fenfluramine Racemate or (+)-Fenfluramine with Neurosteroids that Act at Sigma-1 Receptor

Whether FEN and NOR effects can be related to modulation of endogenous S1R acting hormones was also examined. Neuroactive steroids are endogenous modulators of S1R. Dehydroepiandrosterone sulfate (DHEAS) and pregnenolone sulfate (PREGS) are S1R agonists, and NMDAR activators and GABAR negative modulators. Progesterone (PROG) is a S1R antagonist (Maurice et al., 1998). 3α,5α-tetrahydroprogesterone (allopregnanolone, ALLO) is devoid of S1R activity but is, like PROG, a NMDAR antagonist and GABAR positive modulator. Their role in the excitatory/inhibitory balance in the brain is well known. Before any complex manipulation of endogenous steroid levels (by adrenalectomy/castration and injection of 3β-hydroxysteroid dehydrogenase or 5α-reductase inhibitors to monitor endogenous neurosteroid levels, the effect of FEN and NOR was examined on the behavioral effects induced by exogenously administered neurosteroids, namely DHEAS and PREGS.

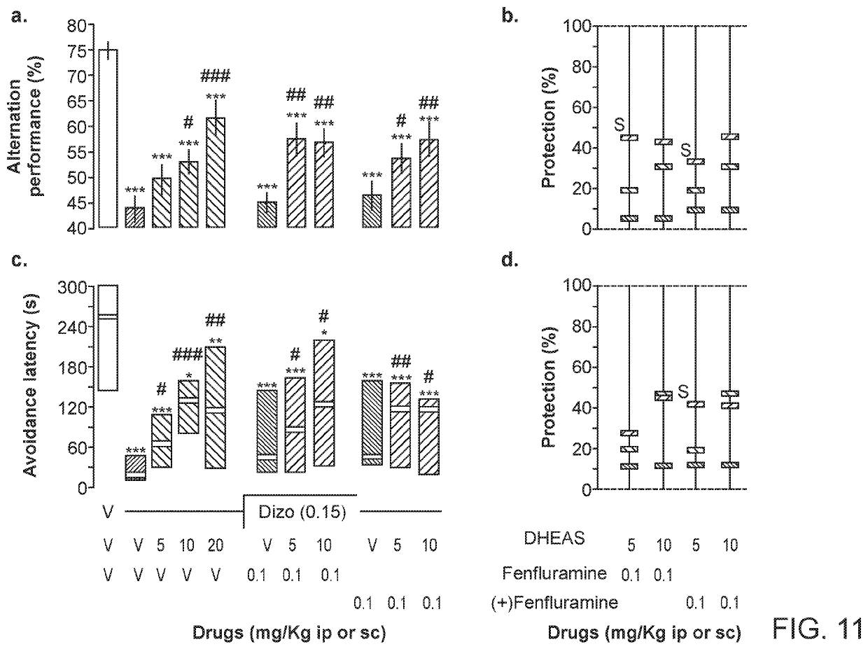

DHEAS was tested alone and in combination with Fenfluramine racemate or (+)Fenfluramine, in Swiss mice administered with dizocilpine (0.15 mg/kg) and tested in the spontaneous alternation test on day 1 and in the passive avoidance test on days 2-3. Results are presented in Tables 5 and FIG. 11.

FIG. 11 and Table 5: Combination studies between DHEAS and Fenfluramine in dizocilpine-treated mice: spontaneous alternation performance in the Y-maze (a, b) and step-through latency in the passive avoidance test (c, d). DHEAS (5-20 mg/kg sc) and/or Fenfluramine or (+)Fenfluramine (0.1-0.3 mg/kg ip) were injected 10 min before dizocilpine (Dizo, 0.15 mg/kg ip), 20 min before the Y-maze test session or the passive avoidance training session, retention being tested after 24 h, without further drug treatment. Data show mean±SEM in (a) and median and interquartile range in (c). In (b, d), protection is shown using a cursor-on-scale representation, with data from V-treated group as 100% and Dizo-treated group as 0%. V: vehicle solution (saline solution or sesame oil for DHEAS). ANOVA: F_{(10,148)}=14.5, p<0.0001, n=11-20 per group, in (a); H=50.6, p<0.0001, n=12-24 per group, in (c). * p<0.05, ** p<0.01, *** p<0.001 vs. V-treated group; #p<0.05, ##p<0.01, ###p<0.001 vs. Dizo-treated group; Dunnett's test in (a), Dunn's test in (c). S: synergistic effect with combination index (CI)<1.

Calculation of the CIs showed that combinations between DHEAS and Fenfluramine racemate or (+)Fenfluramine led to additive or synergistic effects. In particular, the combination between the lowest doses of DHEAS and (+)Fenfluramine was synergistic in both the spontaneous alternation and passive avoidance responses. (See Table 5).

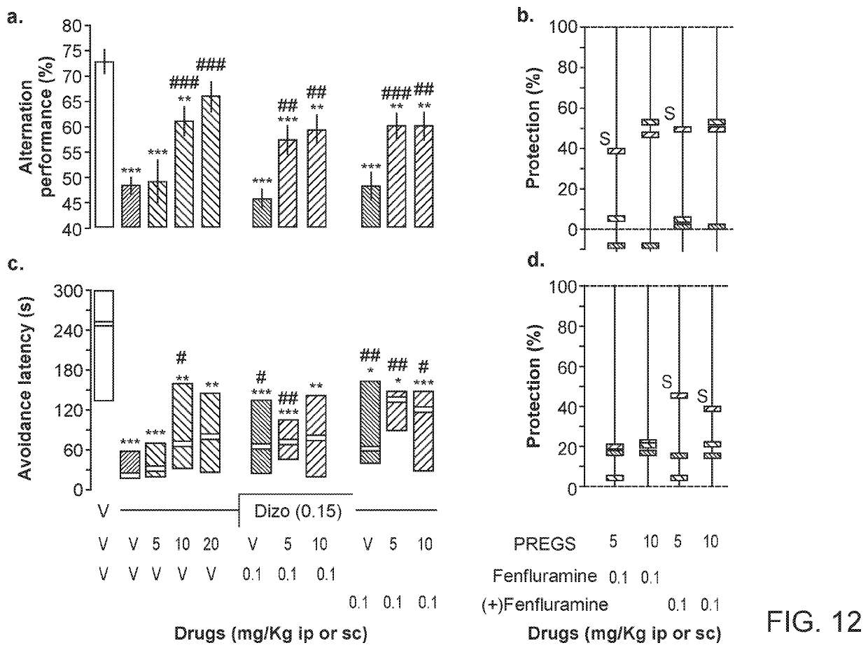

Pregnenolone sulfate (PREGS) was tested alone and in combination with Fenfluramine racemate or (+)Fenfluramine, in Swiss mice administered with dizocilpine (0.15 mg/kg) and tested in the spontaneous alternation test on day 1 and in the passive avoidance test on days 2-3. Results are presented in Table 6 and FIG. 12.

FIG. 12 and Table 6: Combination studies between PREGS and Fenfluramine or (+)Fenfluramine in dizocilpine-treated mice: spontaneous alternation performance in the Y-maze (a, b) and step-through latency in the passive avoidance test (c, d). PREGS (5-20 mg/kg sc) and/or Fenfluramine or (+)Fenfluramine (0.1-0.3 mg/kg ip) were injected 10 min before dizocilpine (Dizo, 0.15 mg/kg ip), 20 min before the Y-maze test session or the passive avoidance training session, retention being tested after 24 h, without further drug treatment. Data show mean±SEM in (a) and median and interquartile range in (c). In (b, d), protection is shown using a cursor-on-scale representation, with data from V-treated group as 100% and Dizo-treated group as 0%. V: vehicle solution (saline solution or sesame oil for PREGS). ANOVA: F(10,143)=11.0, p<0.0001, n=11-20 per group, in (a); H=44.3, p<0.0001, n=10-26 per group, in (c). * p<0.05, ** p<0.01, *** p<0.001 vs. V-treated group; #p<0.05, ##p<0.01, ###p<0.001 vs. Dizo-treated group; Dunnett's test in (a), Dunn's test in (c). S: synergistic effect with combination index (CI)<1.

Calculation of the CIs showed that combinations between PREGS and Fenfluramine racemate led to additive or synergistic effect in the spontaneous alterantion test, but not in the passive avoidance response. PREGS and (+)Fenfluramine combinations led to additive or synergistic effects in all responses, particularly with the lowest dose of the drug. (See Table 6).

TABLE 5
Calculation of combination index (CI) for the DHEAS/Fenfluramine mix.

| | | Cx, | Cx, | |
|---|---|---|---|---|
| Treatment (mg/kg ip or sc) | PP (%) | DHEAS | Fenfluramine | CI |
| (a) YMT (FIG. 7a) | | | | |
| DHEAS (0) | 0.0 ± 7.7 | | | |
| DHEAS (5) | 18.4 ± 9.7 | | | |
| DHEAS (10) | 29.8 ± 7.4 | | | |
| DHEAS (20) | 57.9 ± 11.0¹ | | | |
| Fenfluramine (0) | 0.0 ± 7.7 | | | |
| Fenfluramine (0.1) | 4.4 ± 7.7 | | | |
| Fenfluramine (0.3) | 33.6 ± 5.7² | | | |
| (+)Fenfluramine (0) | 0.0 ± 7.7 | | | |
| (+)Fenfluramine (0.1) | 8.68 ± 8.7 | | | |
| (+)Fenfluramine (0.3) | 40.2 ± 11.3³ | | | |
| DHEAS (5) + Fenfluramine (0.1) | 44.1 ± 10.7 | 15.0 ± 1.3 | 0.40 ± 0.03 | 0.58 ± 0.05 |
| DHEAS (10) + Fenfluramine (0.3) | 42.1 ± 9.2 | 15.3 ± 1.3 | 0.39 ± 0.03 | 0.93 ± 0.08 |
| DHEAS (5) + (+)Fenfluramine (0.1) | 32.2 ± 9.1 | 10.8 ± 1.0 | 0.25 ± 0.02 | 0.87 ± 0.08 |
| DHEAS (10) + (+)Fenfluramine (0.3) | 44.2 ± 10.9 | 15.0 ± 1.3 | 0.34 ± 0.03 | 0.96 ± 0.09 |
| (a) STPA (FIG. 7c) | | | | |
| DHEAS (0) | 0.0 ± 4.0 | | | |
| DHEAS (5) | 19.1 ± 10.7 | | | |
| DHEAS (10) | 47.2 ± 12.8 | | | |
| DHEAS (20) | 40.3 ± 13.3⁴ | | | |
| Fenfluramine (0) | 0.0 ± 4.0 | | | |
| Fenfluramine (0.1) | 11.2 + 12.0 | | | |
| Fenfluramine (0.3) | 65.5 ± 12.1⁵ | | | |
| (+)Fenfluramine (0) | 0.0 ± 4.0 | | | |
| (+)Fenfluramine (0.1) | 11.6 ± 13.6 | | | |
| (+)Fenfluramine (0.3) | 35.2 ± 14.2⁶ | | | |
| DHEAS (5) + Fenfluramine (0.1) | 27.7 ± 13.2 | 9.3 ± 1.2 | 0.14 ± 0.01 | 1.24 ± 0.14 |
| DHEAS (10) + Fenfluramine (0.3) | 44.6 ± 18.0 | 17.7 ± 2.2 | 0.22 ± 0.02 | 1.03 ± 0.12 |
| DHEAS (5) + (+)Fenfluramine (0.1) | 41.2 ± 10.0 | 16.1 ± 2.0 | 0.35 ± 0.04 | 0.59 ± 0.07 |
| DHEAS (10) + (+)Fenfluramine (0.3) | 41.2 ± 11.0 | 16.0 ± 2.0 | 0.35 ± 0.04 | 0.91 ± 0.11 |

Percent protection (PP) was calculated using 100% for V-treated animals and 0% for Dizocilpine-treated animals. YMT: Y-maze test, STPA: step-through passive avoidance, CI: combination index.
C_{x, Drug} was calculated using the linear regression from responses with the drug alone:
¹y = 2.83x + 1.757;
²y = 116.92x − 2.915;
³y = 137.22x − 2.018;
⁴y = 2.02x + 9.013;
⁵y = 225.78x − 4.568;
⁶y = 117.41x − 0.061.

TABLE 6
Calculation of combination index (CI) for the PREGS/Fenfluramine mix.

| | | Cx, | Cx, | |
|---|---|---|---|---|
| Treatment (mg/kg ip or sc) | PP (%) | PREGS | Fenfluramine | CI |
| (a) YMT (FIG. 8a) | | | | |
| PREGS (0) | 0.0 ± 6.8 | | | |
| PREGS (5) | 3.5 ± 17.6 | | | |
| PREGS (10) | 52.4 ± 12.2 | | | |
| PREGS (20) | 72.1 ± 12.4 ¹ | | | |
| Fenfluramine (0) | 0.0 ± 6.8 | | | |
| Fenfluramine (0.1) | -9.81 ± 7.6 | | | |
| Fenfluramine (0.3) | 26.6 ± 9.8 ² | | | |
| (+)Fenfluramine (0) | 0.0 ± 6.8 | | | |
| (+)Fenfluramine (0.1) | 0.4 ± 11.5 | | | |
| (+)Fenfluramine (0.3) | 46.7 ± 8.9 ³ | | | |
| PREGS (5) + Fenfluramine (0.1) | 37.8 ± 11.7 | 10.2 ± 1.2 | 0.45 ± 0.04 | 0.71 ± 0.07 |
| PREGS (10) + Fenfluramine (0.3) | 45.9 ± 11.9 | 12.3 ± 1.5 | 0.53 ± 0.04 | 1.00 ± 0.10 |
| PREGS (5) + (+)Fenfluramine (0.1) | 48.2 ± 10.4 | 12.9 ± 1.6 | 0.33 ± 0.03 | 0.69 ± 0.08 |
| PREGS (10) + (+)Fenfluramine (0.3) | 48.5 ± 10.7 | 12.9 ± 1.6 | 0.33 ± 0.03 | 1.08 ± 0.12 |
| (a) STPA (FIG. 8c) | | | | |
| PREGS (0) | 0.0 ± 4.9 | | | |
| PREGS (5) | 4.4 ± 16.3 | | | |
| PREGS (10) | 21.0 ± 14.6 | | | |
| PREGS (20) | 26.0 ± 19.1 ⁴ | | | |
| Fenfluramine (0) | 0.0 ± 4.9 | | | |
| Fenfluramine (0.1) | 16.8 ± 14.2 | | | |
| Fenfluramine (0.3) | 60.1 ± 13.9 ⁵ | | | |
| (+)Fenfluramine (0) | 0.0 ± 4.9 | | | |
| (+)Fenfluramine (0.1) | 15.6 ± 17.3 | | | |
| (+)Fenfluramine (0.3) | 31.9 ± 16.3 ⁶ | | | |
| PREGS (5) + Fenfluramine (0.1) | 19.6 ± 7.5 | 13.6 ± 1.9 | 0.10 ± 0.01 | 1.33 ± 0.17 |
| PREGS (10) + Fenfluramine (0.3) | 22.4 ± 18.2 | 22.4 ± 2.1 | 0.12 ± 0.01 | 1.49 ± 0.19 |
| PREGS (5) + (+)Fenfluramine (0.1) | 45.3 ± 9.1 | 32.2 ± 4.4 | 0.42 ± 0.05 | 0.39 ± 0.05 |
| PREGS (10) + (+)Fenfluramine (0.3) | 38.7 ± 12.2 | 27.4 ± 3.8 | 0.36 ± 0.05 | 0.65 ± 0.09 |

Percent protection (PP) was calculated using 100% for V-treated animals and 0% for Dizocilpine-treated animals. YMT: Y-maze test, STPA: step-through passive avoidance, CI: combination index.
C_{x, Drug} was calculated using the linear regression from responses with the drug alone:
¹ y = 3.95x − 2.547;
² y = 101.94x − 8.001;
³ y = 166.50x − 6.486;
⁴ y = 1.38x + 0.744;
⁵ y = 202.59x − 1.397;
⁶ y = 102.94x + 2.11.
CI in bold shows synergy.

The instant invention is shown and described herein in a manner which is considered to be the most practical and preferred embodiments. It is recognized, however, that departures can be made therefrom which are within the scope of the invention and that obvious modifications will occur to one skilled in the art upon reading this disclosure.

While the present invention has been described with reference to the specific embodiments thereof, it should be understood by those skilled in the art that various changes can be made, and equivalents can be substituted without departing from the true spirit and scope of the invention. In addition, many modifications can be made to adapt a particular situation, material, composition of matter, process, process step or steps, to the objective, spirit and scope of the present invention. All such modifications are intended to be within the scope of the claims appended hereto.

1.-15. (canceled)

16. A method of treating, or ameliorating symptoms in a patient diagnosed with an epileptic encephalopathy or refractory epilepsy syndrome, comprising:
administering to the patient a therapeutically effective dose of a therapeutic agent consisting essentially of dextrorotatory (+) enantiomer of fenfluramine or a pharmaceutically acceptable salt thereof to the patient,
wherein the amount of (+)-fenfluramine administered is less compared to a therapeutically effective dose of racemic fenfluramine.

17. A method of treating or ameliorating seizures in a patient diagnosed with an epileptic encephalopathy or refractory epilepsy syndrome, comprising:
administering to the patient a therapeutically effective dose of a therapeutic agent consisting essentially of (+) fenfluramine enantiomer or a pharmaceutically acceptable salt thereof to the patient, whereby said seizures are prevented, adjunctively treated or ameliorated.

18. The method of claim 16, wherein the epileptic encephalopathy or refractory epilepsy syndrome is selected from the group consisting of Dravet syndrome, Lennox-Gastaut syndrome, Doose syndrome, Rett Syndrome, West syndrome, Infantile Spasms, and refractory seizures.

19. The method of claim 16, wherein the therapeutically effective dose of (+)-fenfluramine is from about 0.1 mg/kg/day to about 0.8 mg/kg/day.

20. The method of claim 16, wherein the daily dose is selected from the group consisting of 20 mg or less, 10 mg or less, 5 mg or less, and 2.5 mg or less, and wherein the dose is administered in a dosage form selected from the group consisting of forms for oral, injectable, transdermal, inhaled, nasal, rectal, vaginal and parenteral delivery.

21. The method of claim 20, wherein the (+)-fenfluramine oral dosage form is a solution administered dose is in a range from 0.4 mg/kg/day to 0.1 mg/kg/day.

22. The method of claim 20, wherein the (+)-fenfluramine oral dosage form is a solid modified release tablet or capsule.

23. The method of claim 16, wherein the (+)-fenfluramine is for administration as a monotherapy.

24. The method of claim 16, further comprising:
administering one or more of co-therapeutic antiepileptic agents.

25. A method of treating or ameliorating cognitive impairments of memory or learning in a refractory epilepsy or epileptic encephalopathy syndrome comprising an effective dose of racemic fenfluramine or (+)-fenfluramine to a patient in need thereof.

26. The method of claim 25, further comprising:
administering an additional positive modulator of the sigma-1 receptor (S1R) with racemic fenfluramine.

27. The method of claim 25, further comprising:
administering an additional positive modulator of the S1R with (+)-fenfluramine.

28. The method of claim 26, wherein the additional positive modulator of the S1R is chosen from the group consisting of PRE-084, fluvoxamine, ifenprodil, donepezil, sertraline, avanex 2-73, L-687,3834, dextromethorphan, amitriptyline, and dehydroepiandeterone (DHEA).

29. The method of claim 26, wherein the additional positive modulator of the S1R is PRE-084.

30. The method of claim 26, further comprising:
administering a fenfluramine metabolism inhibitor selected from stiripentol and cannabidiol.
